SEQUENCE LISTING (27 sequences)

SEQ ID NO: 1 - DNA, 39 bp, Artificial Sequence
  forward primer to amplify URA3 cassette
gcggcggccgcacgcgtccataccacagcttttcaattc

SEQ ID NO: 2 - DNA, 35 bp, Artificial Sequence
  reversed primer to amplify URA3 cassette
cgctgtacagggtaataactgatataattaaattg

SEQ ID NO: 3 - DNA, 23 bp, Artificial Sequence
  forward primer to determine correct integration in the SIT4 locus at the 3′ side
gtagcgaaatcatgtattgcacc

SEQ ID NO: 4 - DNA, 24 bp, Artificial Sequence
  reversed primer to determine correct integration in the SIT4 locus at the 3′ side and reversed primer to detect correct integration in the SIT2 locus at the 5′ side
ggaaacagctatgacatgattacg

SEQ ID NO: 5 - DNA, 23 bp, Artificial Sequence
  forward primer to determine correct integration in the SIT4 locus at the 5′ side and forward primer to determine correct integration in the SIT4 locus at the 3′ side
tgcacatgttgtccatcaagatg

SEQ ID NO: 6 - DNA, 24 bp, Artificial Sequence
  reversed primer to determine correct integration in the SIT4 locus at the 5′ side
ccaaggcagcggtacatcaagtag

SEQ ID NO: 7 - DNA, 20 bp, Artificial Sequence
  forward primer to determine integration of the MDH3 synthetic construct
cccaaacgctcgtatcttag

SEQ ID NO: 8 - DNA, 20 bp, Artificial Sequence
  reversed primer to determine integration of the MDH3 synthetic construct
tccttgacagcagtgttgac

SEQ ID NO: 9 - DNA, 20 bp, Artificial Sequence
  sets out the sequence of the forward primer to determine integration of the FUMR synthetic construct
aggctgctgacgaagttatc

SEQ ID NO: 10 - DNA, 20 bp, Artificial Sequence
  reversed primer to determine integration of the FUMR synthetic construct
gcaagtgggttctaccaatc

SEQ ID NO: 11 - DNA, 20 bp, Artificial Sequence
  forward primer to determine integration of the SpMAE1 synthetic construct
gctccacacgttccattgtc

SEQ ID NO: 12 - DNA, 20 bp, Artificial Sequence
  reversed primer to determine integration of the SpMAE1 synthetic construct
tatggtctggaacccatagc

SEQ ID NO: 13 - DNA, 23 bp, Artificial Sequence
  forward primer to determine correct integration in the SIT2 locus at the 5′ side
tgatcttgtagaaagtaccgagg

SEQ ID NO: 14 - DNA, 25 bp, Artificial Sequence
  reversed primer to determine correct integration in the SIT2 locus at the 3′ side
ctttgttcttccgttatgtcaacac

SEQ ID NO: 15 - DNA, 21 bp, Artificial Sequence
  forward primer to determine integration of the PCKa synthetic construct
ggttgttacgccaagaccatc

SEQ ID NO: 16 - DNA, 21 bp, Artificial Sequence
  reversed primer to determine integration of the PCKa synthetic construct
gcaagacaccgaaagcatcag

SEQ ID NO: 17 - DNA, 21 bp, Artificial Sequence
  forward primer to determine integration of the FRDg synthetic construct
gaaagaccagacggtaccaag

SEQ ID NO: 18 - DNA, 20 bp, Artificial Sequence
  reversed primer to determine integration of the FRDg synthetic construct
tttgagcagcagcgttcaag

SEQ ID NO: 19 - DNA, 25 bp, Artificial Sequence
  forward primer to determine correct replacement of the ADH1 locus by the URA3 PCR fragment and PYC2 synthetic construct at the 5′ side
gaacaatagagcgaccatgaccttg

SEQ ID NO: 20 - DNA, 20 bp, Artificial Sequence
  reversed primer to determine correct replacement of the ADH1 locus by the URA3 PCR fragment and PYC2 synthetic construct at the 5′ side
gcacagaacaaaaacctgca

SEQ ID NO: 21 - DNA, 20 bp, Artificial Sequence
  forward primer to determine correct replacement of the ADH1 locus by the URA3 PCR fragment and PYC2 synthetic construct at the 3′ side
agcctcatcagccttcaatc

SEQ ID NO: 22 - DNA, 20 bp, Artificial Sequence
  reversed primer to determine correct replacement of the ADH1 locus by the URA3 PCR fragment and PYC2 synthetic construct at the 3′ side
aatggaatttgggcggaggg

SEQ ID NO: 23 - DNA, 20 bp, Artificial Sequence
  forward primer to determine integration of the PYC2 synthetic construct
tcgatgtcgccatgagattc

SEQ ID NO: 24 - DNA, 20 bp, Artificial Sequence
  reversed primer to determine integration of the PYC2 synthetic construct
accagccatgtccttgatac

SEQ ID NO: 25 - DNA, 18320 bp, Artificial Sequence
  plasmid pSUC044
atttcgcgcaagctaattccctattgtgagtcgtattaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagaggtggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagactaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttggtcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagctggcgaaagggggatgtgctgcaaggcgattaagttgggtaaggccagggttttcccagtcacgacgttgtaaaacgacggccagtgccaagctagctttgcagtgcgaatgggggaggagagtagattctttcttcaagaaagcaaaatattgaaatcttaagagaactagatgaaagatttgatttattaacgaaccggaaggttatatactattcccattttttgaaggtatatgctaatatgtttgagcgtttgttatttccccactcgtctttttgcctaaatagtgcctcgaaacctaaagtggatttgtcattagctgtgacttacatttccgaacttatccgctcaactagaagtcaatggagaagcaatgcattaaattgcctttcgccctctctctcgagttcctttggatcaccctgatgcacgccttgcatatcgagaaatctcttcgaaggcatcttacagaattttcgtttccttttctctatttttcattctgagaaatatttgcctgcatacattgtaatggcgatctggtgatccgcaattctagaacacacaatatgcatgtaatcgctgattttttttgttttagaagctctatcttcaggtaaaaatgagtagagaaaaaaaaacatactggatcgatgcagaattagggggttattatcctgcaggtacatgattttcagtgggaacattgctttttagtagtccggttctcaacaacttgtctaagtgttgaaaacaaaagaaatggcgtagaaacaaagtagtgtaagtaaatctgccaatgttctatgtataaaaagtaaaggcaagaagaggttctatgcatatttctgaaaatatctaatacactattataatgcatcaagaaactgtcgtatgatgaagtgcctatgagtttttgtgtacgtgcttctctagtatgtagccggttttctctttttacctctttttactacttatactactacttttactacctttcttccacgtaatctagatctcaagccacaattcttgccctatgctccaacgtatacaacatcgaagaagagtctttctttagggagtcattggaaaagatagtatgatggtattcgatttacctatgtcgcaaaagaaagtccggggcaacaccacagaatgctttctctgtactaataacctgttgtgcgcttaacggtctaatcgttaatcagcggtggttaaatttttgtaaatctaatgttccatgattttctttcttcaaaaggaacatgtagcgaaaatcttttttttactttgatacactgcaattgtttctgagcatgctgaaattttctcgatgttttttttttttattggcatccaagtaattaatccttatgctacgaaaaagttgtaggaatgaatcatgcataatctaacggatatcatcatatactctgtgctaatattctaaacaagttcgaaaatattttcttggcccatgtaataggtggtaagtgtattgctttgataggaacgtcattatcgcacaagacaatcggcactaataaccgtttaaatattatcatgcatgtatacatcagtatctcatagaaatatacctgtaagtacatacttatctaagtataaattctcgacctatggagtcaccacatttcccagcaacttccccacttcctctgcaatcgccaacgtcctctcttcactgagtctccgtccgataacctgcactgcaaccggtgccccatggtacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggaatcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccatggctcctgaccgaggacggatttggtgaagaggcggaggtcctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagcccttgactcgaagctggtctttgagagagatggggaggccatggagtggaccaacgggtctcttgtgctttgcgtagtattcatcgagttcccttgcctgcgcgagagcggcgtcagggaagaactcgtgggcgcagtttgttaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattgtatatgagatagttgattgtatgcttggtatagcttgaaatattgtgcagaaaaagaaacaaggaagaaagggaacgagaacaatgacgaggaaacaaaagattaataattgcaggtctatttatacttgatagcaaagcggcaaactttttttatttcaaattcaagtaactggaaggaaggccgtataccgttgctcattagagagtagtgtgcgtgaatgaaggaaggaaaaagtttcgtgtgttcgaagatacccctcatcagctctggaacaacgacatctgttggtgctgtctttgtcgttaattttttcctttagtgtcttccatcattttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaagaggaagtccatattgtacacccggaaacaacaaaaggatggtaccctggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgactgtgaggggagcgtttccctgctcgcaggtctgcagcgaggagccgtggtaccggttactcaagcacaaactcccaacactagagtccactcagctccaattcccttacaaactcaatataacaaaaacagagcagaaaacggtcaccactcctatggttctccccaaagttattctccaagacatacgaaaacacctgtggatcctagatataatgttatcgcacagaaaccagcaggcaggcctatacctccagcgccaacccattataacaacttgaacacttccgctcaacggatagcttcctctcctcctcccctaattcacaatcaagcagtgcctgcacaactcttgaagaaagttgcacctgcttcgttcgatagcagagaagatgtacgagacatgcaagtggccacacagctatttcataaccatgatgtaaagggcaaaaaccgactgacagctgaggaactacagaacttactacaaaacgacgacaactcccatttttgtatatcatcagtagatgcgctgataaatttatttggtgcttccaggtttggcactgtcaaccaggcagaattcatcgccctatacaaaagagtgaaaagttggagaaaagtttatgtggacaatgatatcaacggatcgctcaccatttctgtaagcgaatttcataactcacttcaagaactaggatatctaataccttttgaagttagcgagaaaacatttgaccaatatgctgagtttataaacagaaatggaacaggaaaagaactaaagtttgataaattcgttgaggcgttagtttggctaatgagattaacaaaattattcaggaaattcgatactaatcaagaaggcattgcaaccatacagtacaaagattttatctatgctacattatatttaggtcgtttcctacctcattgatgaaaaccattcgttctttctggtcgtaatacaaatagaagaggtaaaccaatcaatggcccgttagtttgccatttgctgcatccttcccatgcaaagtgtcttcgtatttagtgatgttttgttagcgacacaaaagagacctcgatgacagacatttttttttctttacttaatgtattataagtgtgtctgacctcgactatcattataatattaatttgaagttctattttacatttatatgagttattccattacataataaggatatcaagagcagattagtgtcttttagattatacatctttttccccccttttttgttgttggtagtggaaagaaaaggatcaattaacagaaataaataaataaataataaaaacctaaaattccttttgcgtcattgaatttttattatgaagtcaaaagggagtcggttgtcaacagactgtcctgtcgaatttcccaagatagtctctggattcgctgaggaagtgaaaatacgtagacaaagttcccaaggacagtacgccgtcgattcacatcctccgaaaagccctgaactgaaacacagaagacacgcgtggccggccttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggttgatggtagatcttctgcttccattgttgccgttgacccagaaagagctgccagagaaagagatgctgctgccagagctttgttgcaagactctccattgcacaccaccatgcaatacgctacctctggtttggaattgactgttccatacgctttgaaggttgttgcttctgctgacactttcgacagagccaaggaagttgctgatgaagtcttgagatgtgcctggcaattggctgacaccgttttgaactctttcaacccaaactctgaagtctctttagtcggtagattaccagtcggtcaaaagcatcaaatgtctgctccattgaaacgtgtcatggcttgttgtcaaagagtctacaactcctctgctggttgtttcgacccatccactgctccagttgccaaggctttgagagaaattgctttgggtaaggaaagaaacaatgcttgtttggaagctttgactcaagcttgtaccttgccaaactctttcgtcattgatttcgaagctggtactatctccagaaagcacgaacacgcttctttggatttgggtggtgtttccaagggttacatcgtcgattacgtcattgacaacatcaatgctgctggtttccaaaacgttttctttgactggggtggtgactgtcgtgcctccggtatgaacgccagaaacactccatgggttgtcggtatcactagacctccttccttggacatgttgccaaaccctccaaaggaagcttcttacatctccgtcatctctttggacaatgaagctttggctacctctggtgattacgaaaacttgatctacactgctgacgataaaccattgacctgtacctacgattggaaaggtaaggaattgatgaagccatctcaatccaatatcgctcaagtttccgtcaagtgttactctgccatgtacgctgacgctttggctaccgcttgtttcatcaagcgtgacccagccaaggtcagacaattgttggatggttggagatacgttagagacaccgtcagagattaccgtgtctacgtcagagaaaacgaaagagttgccaagatgttcgaaattgccactgaagatgctgaaatgagaaagagaagaatttccaacactttaccagctcgtgtcattgttgttggtggtggtttggctggtttgtccgctgccattgaagctgctggttgtggtgctcaagttgttttgatggaaaaggaagccaagttgggtggtaactctgccaaggctacctctggtatcaacggttggggtactagagctcaagctaaggcttccattgtcgatggtggtaagtacttcgaaagagatacctacaagtctggtatcggtggtaacaccgatccagctttggttaagactttgtccatgaaatctgctgacgctatcggttggttgacttctctaggtgttccattgactgttttgtcccaattaggtggtcactccagaaagagaactcacagagctccagacaagaaggatggtactccattgccaattggtttcaccatcatgaaaactttagaagatcatgttagaggtaacttgtccggtagaatcaccatcatggaaaactgttccgttacctctttgttgtctgaaaccaaggaaagaccagacggtaccaagcaaatcagagttaccggtgtcgaattcactcaagctggttctggtaagaccaccattttggctgatgctgttatcttggccaccggtggtttctccaacgacaagactgctgattctttgttgagagaacatgccccacacttggttaacttcccaaccaccaacggtccatgggctactggtgatggtgtcaagttggctcaaagattaggtgctcaattggtcgatatggacaaggttcaattgcacccaactggtttgatcaacccaaaggacccagccaacccaaccaaattcttgggtccagaagctctaagaggttctggtggtgttttgttgaacaaacaaggtaagagatttgtcaacgaattggatttgagatctgttgtttccaaggccatcatggaacaaggtgctgaatacccaggttctggtggttccatgtttgcttactgtgtcttgaacgctgctgctcaaaaattgtttggtgtttcctctcacgaattctactggaagaagatgggtttgttcgtcaaggctgacaccatgagagacttggctgctttgattggttgtccagttgaatccgttcaacaaactttagaagaatacgaaagattatccatctctcaaagatcttgtccaattaccagaaaatctgtttacccatgtgttttgggtaccaaaggtccatactatgtcgcctttgtcactccatctatccactacaccatgggtggttgtttgatttctccatctgctgaaatccaaatgaagaacacttcttccagagctccattgtcccactccaacccaatcttgggtttattcggtgctggtgaagtcaccggtggtgtccacggtggtaacagattaggtggtaactctttgttggaatgtgttgttttcggtagaattgccggtgacagagcttctaccattttgcaaagaaagtcctctgctttgtctttcaaggtctggaccactgttgttttgagagaagtcagagaaggtggtgtctacggtgctggttcccgtgtcttgagattcaacttaccaggtgctctacaaagatctggtctatccttgggtcaattcattgccatcagaggtgactgggacggtcaacaattgattggttactactctccaatcactttgccagacgatttgggtatgattgacattttggccagatctgacaagggtactttacgtgaatggatctctgctttggaaccaggtgacgctgtcgaaatgaaggcttgtggtggtttggtcatcgaaagaagattatctgacaagcacttcgttttcatgggtcacattatcaacaagctatgtttgattgctggtggtaccggtgttgctccaatgttgcaaatcatcaaggccgctttcatgaagccattcatcgacactttggaatccgtccacttgatctacgctgctgaagatgtcactgaattgacttacagagaagttttggaagaacgtcgtcgtgaatccagaggtaaattcaagaaaactttcgttttgaacagacctcctccattatggactgacggtgtcggtttcatcgaccgtggtatcttgaccaaccacgttcaaccaccatctgacaacttattggttgccatctgtggtccaccagttatgcaaagaattgtcaaggccactttaaagactttaggttacaacatgaacttggtcagaaccgttgacgaaactgaaccatctggaagttaaggtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggatgggcccggcgcgccgcggccgcgagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaacaaaaaatgactgatttgaacaaattggtcaaggaattgaatgatttgggtttgactgacgtcaaggaaattgtctacaacccatcttacgaacaattattcgaagaagaaaccaagccaggtttggaaggtttcgacaagggtactttgaccactttaggtgctgttgctgttgacaccggtattttcaccggtcgttctccaaaggacaaatacattgtttgtgatgaaaccaccaaggacaccgtctggtggaactctgaagctgccaagaacgataacaagccaatgactcaagaaacctggaaatctttgagagaattggttgccaagcaattgtctggtaagagattattcgttgttgacgctttctgtggtgcttctgaaaagcacagaattggtgtcagaatggtcactgaagttgcttggcaagctcatttcgtcaagaacatgttcatcagaccaactgacgaagaattgaagaacttcaaggctgacttcaccgttttgaatggtgccaagtgtaccaacccaaactggaaggaacaaggtttgaactctgaaaactttgttgctttcaacatcactgaaggtatccaattgattggtggtacctggtacggtggtgaaatgaagaagggtatgttctccatgatgaactatttcttgccattgaaaggtgttgcttccatgcactgttctgccaatgtcggtaaggatggtgacgttgccatcttcttcggtctatccggtactggtaagaccactctatccactgacccaaagagacaattgattggtgatgacgaacacggttgggacgaatctggtgtctttaactttgaaggtggttgttacgccaagaccatcaacttatctcaagaaaacgaaccagatatctacggtgccatccgtcgtgatgctttgttggaaaacgttgttgtcagagctgacggttctgttgacttcgacgacggttccaagactgaaaacaccagagtttcttacccaatctaccacattgacaacattgtcagacctgtttccaaggctggtcacgctaccaaggttatcttcttgactgctgatgctttcggtgtcttgccacctgtttccaaattgactccagaacaaaccgaatactacttcttgtccggtttcactgccaaattggctggtactgaaagaggtgtcactgaaccaactccaactttctctgcttgtttcggtgctgctttcttatctttgcacccaatccaatacgctgatgtcttggttgaaagaatgaaggcttctggtgctgaagcttacttggtcaacaccggttggaacggtaccggtaagagaatctccatcaaggataccagaggtatcattgatgctatcttggacggttccattgaaaaggctgaaatgggtgaattgccaatcttcaacttggccattccaaaggctttgccaggtgttgacccagccatcttagatccaagagacacctacgctgacaaggctcaatggcaagtcaaggctgaagatttggctaacagattcgtcaagaactttgtcaaatacactgctaacccagaagctgccaaattggttggtgctggtccaaaggcttaagcgtacgcaattccggggaattgtcattctaatattttatccacacacacaccttaaaatttttagattaaatggcatcaactcttagcttcacacacacacacacaccgaagctggttgttttatttgatttgatataattggtttctctggatggtactttttctttcttggttatttcctattttaaaatatgaaacgcacacaagtcataattattctaatagagcacaattcacaacacgcacatttcaactttaatatttttttagaaacactttatttagtctaattcttaatttttaatatatataatgcacacacactaatttattcattaattttttattgagtaggatttgaaaatatttggtatctttgcaagatgtttgtatagagggacaaagaatcgtctttattatggtcaaggctttacgtcataatagttcctgcccagctcttctataatactttaaagatctcttctcgtttgctccatttggaagtctcgcttacgtttatgcgcccatacagacactcaagatacacacttacatgaacgtatacaaatttactaacactacttgaaaatatgaaccacagtacatcatattaagacgtagtattcgatgattgaaggccgcctccgcgaaatacctttactgattttgccggttaatcgcatcgaaatttcttcatcacaagaaagcaaacaaatcgccaggccattctacaagtttccttttcttatgaagatgtaaaagctactaaggcgtcattactctagatgactcagtttagtctgaccttctatagtatactaccctggcgctatgatgatgagcggttcttttattgcggaaacgaaaattccgggaccggcgaaatttgcccggttttgtccgtaaccggcttcatgagtcggcttcaatagtagttgaatacttatttaaacagcagaacttaactcactcatcacgctgtttccgctgaattttctcaaaatatctaagcagtcaacaaatataaagaatattgaaattgacagtttttgtcgctatcgatttttattatttgctgttttaaatcatggtttacactccatccaagggtccaagaactctttacgataaggtttttgatgcacatgttgtccatcaagatgaaaatggttcctttttgttgtatatcgacagacacttggttcatgaagtcacctctccacaagcttgtcgacggatcccgggctgcagtctagaaactatattgatgaagaaataagatcgtacattttccaagaagaacaacctgataggacaaaaatggttgataatagagtccattgttgtttgtactttctgagaccttcaaataagggaattgatactttagacgtcgtaacaatgaaaaaattagcgaagagagtgaatttaatcccggttattgctaaatcagatttgctaacgaaagaggaattgaaaaacttcaaaacacaagttagagaaataataagagtacaagatatccctgtatgtttctttttcggcgatgaagttttgaatgcaacacaagatatttttcaaaaatatccattcagtataattgcatctaatgagtacatttttaatgaaaagggcgaaaaagttaaaggaagacaatacaaatggggcgctgttgacattgaaaatgaaaagtactgtgacttcaaaatcttgcaaaagacgatttttgattggaatttaattgatcttgtagaaagtaccgaggattattatgaaaaatgcagatcggccatcttggccgatctgaaatgctaagaactaggctattaaaggccagagattgcttaacaacgaaaagtgttgacataacggaagaacaaaggaaatttttggaggaagaaatgaacttcgatgaaatcgaggaaaacaaactcaaaaattacaagtgctatgaaataattaataaaacggtcatggataaggtggctacagaatgggatcctgaatttataactagacaattagaagctaagaaaaaattcaacgagctgtccaacagagaaatttcaaaatttcgagactggaaaaagagcctattcatggaacaagagaattttaaccaagagattgaacaattgaatcacaagttggaaaacttacaactggaatgtcaggacttggaatacaagctgttaatcggaaaaagttccaacagccattccacagatagtgctactttagtaaacgttcacatcaaaaggtagtattaattaaaaaaaaaaaaaagccaata

SEQ ID NO: 26 - DNA, 18127 bp, Artificial Sequence
  plasmid pSUC047
catcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagatttaaagtaaattcaccttaactgtccaagatgaaagacttacccttttcaatgttctttctcaattccttgacagcagtgttgaccaattgttcttctcttggagacaatttttccaaaacagaagtgtcaatggaaacaacggaaccgtttctcaaaacaattggcaaagagaagtattcaatggagttgtcaccgactaattgttgagccttcttaccgttcttcaaacctggcaagtagacgaaagcagacaaagattcagtttctggcttttcgttgtggaaagaacgtaagacttcttcagcaaatttggcaccagcgaaagccatggacaaggtagcagaaccggcaccttgcttggccttgacaatttcgtcaccaccgaattggactctgtggatgaaatgttcgtattgcttgtccaattggaaaaccaaggatttgtcagtgatgattggaatgatggtttcaccagagtgaccaccgatgacggtgacctttctgtgcatggtggtcttgtcttgttcttgaccgatctttgggttcttcaacatcaagtagtcaaccaagaaagtttcagctctgaccaaatccaagttggtgacacccataacgttacctggcttgaacttacccatcttcttcaaagtttcaacggcaattggaaccaaagagttaacagggttggagatgactaagatacgagcgtttggggcaaatttaccgacagcagtaaccaaagacttaacgataccagcgttcatcttgaacaaatcatctctggtcaaacctggctttcttggaacaccagctggaatcaagacaacttgagcattggacaaagtgttttcgatggaatccttgtcgtaaccaacacaagaggagttggtgttgatgtgggacaaatccttaccaataccttcagcagctctgatatcgtacaaagccaattcagaaacgtatggagacaatttcaataatagagataatggttgaccgacaccaccagaagcacctaagatggcaaccttaaccattttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaggccggccacgcgtgaagatctcgttatgtacccgaatatgtcagtttacattggtcagtctattggagaattaagtttgatcgtaggtatagaccggacaatatgccggaatatgtaaggcaattgttccaagatttggaaggtattgatttaaaaagtaataaagtttcaaataaatatgataagcaagataatagcaacgggagtgaaatcaatgggggcttttttgataatgaggaagggcaggaactccacatgggtcaaaaagcaagttattttgcaacgacatacaattcaagattatttgacagtaaatactcccaattaaaaaagaaattcatggactgggatagtaattcctggacagatattccagatgatttaaaaatatacctacagcaagatgaatcgctttagcattaaaaaaaccccttcggtacgtaatataaaaaattttataggtaatatacatatataaaaatacttcaatcatttttacaatcttgtatactttatacaacatgtgaaatcttctgcttctggacatcaatattcaaatacaggccaatcttaggtaaaacatttggagaaaagaaggataaggcaggacgagggaagataaatagtttcgttaattataaatacatgcagataaataaaggaatatcaaatattatgaatagaaaaagaagatggtgagacaaaaaagtagtaataaataggtccaaatcttctttatttcccctttcttttcttatccttttgttttctccatattgtataagaatatattcttaggaaaatcaacagggaatacagtatagtgattttcgttcctttttgagcgtaatcccttcgagactgtgatgttgattatttttgttgtgatttcaaaattcttaggttagttgtatagttcccgttcataacataatggatagtaaatgaaaaatcaaaataagggtgaaacaaatagacaataaagatgtagttttcgaggacgaaaaacaaacctaaccaacaatgaccttatcaccatcgaattcataagcaggaatttctaagtttaagggggcaggtccctttctgattctaccggaaatatcataatgtgaaccatggcaaggacagaaccaaccaccaaaatcaccggcttcaccaattggaacacaacctaagtgagtacaaatacccagcataattaaccattgagggtctttgactctgtcagcatcggtctgtgggtccttcaaagcggacatatccacactgttggcttcctgaatttcatgaggagttctgtgtctaatgaacacaggcttaccttgccatttgacaaccacgtttttacccaatgggatagccgctaaattaacttcaactttagccatagccaaaacatcggcagtagcggtcatagatgaaataaaggtttctacggttgatttggcacctgcagatgacaaaagacccatagcaccgaccataaagtaagcataagaacggcctttatcagcatcgttattttcctttaaaacgtcatcaaaatttggggtcctgtacgtggatttgctagccagcaaagattgagaaatcaggtaccacggctcctcgctgcagacctgcgagcagggaaacgctcccctcacagtcgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagggtaccatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacacaaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactatctcatatacaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttaacaaactgcgcccacgagttcttccctgacgccgctctcgcgcaggcaagggaactcgatgaatactacgcaaagcacaagagacccgttggtccactccatggcctccccatctctctcaaagaccagcttcgagtcaagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgaggacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccataggtcgagaatttatacttagataagtatgtacttacaggtatatttctatgagatactgatgtatacatgcatgataatatttaaacggttattagtgccgattgtcttgtgcgataatgacgttcctatcaaagcaatacacttaccacctattacatgggccaagaaaatattttcgaacttgtttagaatattagcacagagtatatgatgatatccgttagattatgcatgattcattcctacaactttttcgtagcataaggattaattacttggatgccaataaaaaaaaaaaacatcgagaaaatttcagcatgctcagaaacaattgcagtgtatcaaagtaaaaaaaagattttcgctacatgttccttttgaagaaagaaaatcatggaacattagatttacaaaaatttaaccaccgctgattaacgattagaccgttaagcgcacaacaggttattagtacagagaaagcattctgtggtgttgccccggactttcttttgcgacataggtaaatcgaataccatcatactatcttttccaatgactccctaaagaaagactcttcttcgatgttgtatacgttggagcatagggcaagaattgtggcttgagatctagattacgtggaagaaaggtagtaaaagtagtagtataagtagtaaaaagaggtaaaaagagaaaaccggctacatactagagaagcacgtacacaaaaactcataggcacttcatcatacgacagtttcttgatgcattataatagtgtattagatattttcagaaatatgcatagaacctcttcttgcctttactttttatacatagaacattggcagatttacttacactactttgtttctacgccatttcttttgttttcaacacttagacaagttgttgagaaccggactactaaaaagcaatgttcccactgaaaatcatgtacctgcaggataataaccccctaattctgcatcgatccagtatgtttttttttctctactcatttttacctgaagatagagcttctaaaacaaaaaaaatcagcgattacatgcatattgtgtgttctagaattgcggatcaccagatcgccattacaatgtatgcaggcaaatatttctcagaatgaaaaatagagaaaaggaaacgaaaattctgtaagatgccttcgaagagatttctcgatatgcaaggcgtgcatcagggtgatccaaaggaactcgagagagagggcgaaaggcaatttaatgcattgcttctccattgacttctagttgagcggataagttcggaaatgtaagtcacagctaatgacaaatccactttaggtttcgaggcactatttaggcaaaaagacgagtggggaaataacaaacgctcaaacatattagcatataccttcaaaaaatgggaatagtatataaccttccggttcgttaataaatcaaatctttcatctagttctcttaagatttcaatattttgctttcttgaagaaagaatctactctcctcccccattcgcactgcaaagctagcttggcactggccgtcgttttacaacgtcgtgactgggaaaaccctggccttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagaccaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttagtctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccacctctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttaatacgactcacaatagggaattagcttgcgcgaaattattggcttttttttttttttaattaaaagaaaacattctctagggattacgaggtaaagatacattttcaaggcttattcgattctgtgaactcagttggaatattaagggacaggttgtttccttgcacccagagaagcaatatcgttgagcatgttcgacattgcgtatccttggatgaaagacgtggaaaattcaagcagttatgtttcactccgatgccgtacattccgaaactattttcattgacatattgtaatcatataactgaccagtgttcgccggtgccaacttctaatgcattaatgcgtgatctaaccccggaaaatcctttgataaaatacactttaaaaagtggcgcacattctattagtaatccttctccactcattcctgataaccctggaaggttgttatcgagcaaaagcgaggaaactacagagttgctgttggacctgaactcattcttagaaggtaattcatacgcgagagatacagaatgttcaacaagaggaattgaagccattttccaacttcaatctatccaaggcagcggtacatcaagtagaatgactatgacacccgacttgattgaaaaatggtttccaggtgatcggccatcttggccgatcattctgacgttggtggaggttgggcgcctgactgtgagacagaagagaacttgtcaaatttaacgctgcgatggattttagcagaggcaatcaaatttggtgttaaattcaaacctggtgcaatacatgatttcgctaccaaacacacttcgattggatctttattcgcagacacacatgattaccttagtttcaactcaccaaagaaatgttccctactaggagtgagtgataatgaggatggagcccgagaggataaatctggcagaaatgagagaatggaagattgtctaaaaaatataaaagagactagattgagcttgaaagatgaaaaagaaaaagtgaaggatgcttttactcttaaatgtggacatgcaaataaatttatgagattggtgtggtgggtattggaactgctccccattggaatacgaatggaaaataaagaaggaaagtggcaaaattttcatacacctaacctcggaagatcgtcgacaagcttgtggagaggtgacttcatgaaccaagtgtctgtcgatatacaacaaaaaggaaccattttcatcttgatggacaacatgtgcatcaaaaaccttatcgtaaagagttcttggacccttggatggagtgtaaaccatgatttaaaacagcaaataataaaaatcgatagcgacaaaaactgtcaatttcaatattctttatatttgttgactgcttagatattttgagaaaattcagcggaaacagcgtgatgagtgagttaagttctgctgtttaaataagtattcaactactattgaagccgactcatgaagccggttacggacaaaaccgggcaaatttcgccggtcccggaattttcgtttccgcaataaaagaaccgctcatcatcatagcgccagggtagtatactatagaaggtcagactaaactgagtcatctagagtaatgacgccttagtagcttttacatcttcataagaaaaggaaacttgtagaatggcctggcgatttgtttgctttcttgtgatgaagaaatttcgatgcgattaaccggcaaaatcagtaaaggtatttcgcggaggcggccttcaatcatcgaatactacgtcttaatatgatgtactgtggttcatattttcaagtagtgttagtaaatttgtatacgttcatgtaagtgtgtatcttgagtgtctgtatgggcgcataaacgtaagcgagacttccaaatggagcaaacgagaagagatctttaaagtattatagaagagctgggcaggaactattatgacgtaaagccttgaccataataaagacgattctttgtccctctatacaaacatcttgcaaagataccaaatattttcaaatcctactcaataaaaaattaatgaataaattagtgtgtgtgcattatatatattaaaaattaagaattagactaaataaagtgtttctaaaaaaatattaaagttgaaatgtgcgtgttgtgaattgtgctctattagaataattatgacttgtgtgcgtttcatattttaaaataggaaataaccaagaaagaaaaagtaccatccagagaaaccaattatatcaaatcaaataaaacaaccagcttcggtgtgtgtgtgtgtgtgaagctaagagttgatgccatttaatctaaaaattttaaggtgtgtgtgtggataaaatattagaatgacaattccccggaattgcgtacgcttaatccttggcagaaatcatgtcctcaggtctaacccattggtcgaattcttcagaggtcaagtaacccaaagatagagcagcttctttcaaagtggtaccttccttgtgagccttcttggcacacttggcagccttgtcgtaaccaatgtgagggttcaaagcagtgaccaacatcaaagattcgttcatgatggaggagatcttcttttcgttagcttcaataccgacaacacagttcttggtgaaagagatggaagcgtcagagattaatctgatggattggatcaagttcttgatcatgactggtttaaagacattcaattcgaattgaccgttggaaccagcaacagagatggcagtgttgttacccatgacttgagcacaaaccatggtcatagcttcacattgagttgggttgaccttacctggcatgatggaagaacctggttcgttttctggtagagacaattcacctaaaccacatcttggaccagaacccaagtaacggatatcgttggcaatcttcatcaaagaacaagcaacggtgttcaaagcaccgtgagcttcaaccaaagcgtcgtgagcagccaaagcttcgaatttgtttggagcggtcttgaatggtaaaccagtgatggaagcaatggcttcagcaaccttggcatcgaaaccctttctggtgttcaaaccagtaccgacagcagtaccaccttgagccaagttgtataatctttccaaagtaccttgaacacgagcaataccgtaggtcaattgttgagtgtaaccggagaattcttgacctaaagtcaatggggtagcatcttgcaagtgggttctaccaatcttgatgatgtgttcgaattcagcagatttggcttgcaaagcatctctcaaagtggtcaaagctggaatcaatctaccgtgaatttcaacaacggcagcaacgtgcatggcagttgggaaagtgtcgttggaagattgagacatgttgacatgatcgtttgggtggactggagccttggaacctaattcaccacccaacaattcaatggctctgttggagatgacttcattgacgttcatcttggtttgagtaccagaaccggtttgccagacaaccaatgggaaatggtcaatcaaagaaccatcgataacttcgtcagcagccttttggatggcttcaccaacctttgggtccaaaccgtaggtcatgttgacggtggcagcagccttcttcaaaacaccgaaagctctgattaatggttctggcattctttcagttggaccaccaatgtcaaagttttgcaaagatctttgagtttgagcaccccagtaacggtcagctggaacttgcaagtcaccgaaggtatctctttcagctctgaatttttgcaaagcagcagaagcagaggacattttttgtttatgtatgtgttttttgtagttatagatttaagcaagaaaagaatacaaacaaaaaattgaaaaagattgatttagaattaaaaagaaaaatatttacgtaagaagggaaaatagtaaatgttgcaagttcactaaactcctaaattatgctgccctttatattccctgttacagcagccgagccaaaggtatataggctcctttgcattagcatgcgtaacaaaccacctgtcagtttcaaccgaggtggtatccgagagaattgtgtgattgctttaattaatttcggagaatctcacatgccactgaagattaaaaactggatgccagaaaaggggtgtccaggtgtaacatcaatagaggaagctgaaaagtcttagaacgggtaatcttccaccaacctgatgggttcctagatataatctcgaagggaataagtagggtgataccgcagaagtgtctgaatgtattaaggtcctcacagtttaaatcccgctcacactaacgtaggattattataactcaaaaaaatggcattattctaagtaagttaaatatccgtaatctttaaacactatgtagttaggtctcgcggccgcggaggaaatgagaaatgagaggtatgtaaatagaaatagactagctccacttttaagaattatttatgcaattaaatacatgggtgaccaaaagagcgggcggatacccgcgtcaccacaagcagaataaaaggtaaacctgaaattgttttaacataaaatgaaaaatgcttgtttgcaaccctatatagaatcataaaacattcgtgactataaaatgaataaactaaactattctaagaaaatgaaataaatgacaaaaaaacgtgttttttggactagaaggcttaatcaaaagctcttaaacgctttcgtgttcagaagatggaggatcagattcaccaccagtggaagtaacgtgggtgtcaaccttttccaaagaagctggagccttttctggtgggaaagttgggttcaaaacaccagtgtttggctttggaggtgggtgagcatcttcatccttacctgggtaacacaaatcgttgaccaagaaagctctgaccatcaagtacatcaacaagatccattggatacataggatgacaccgatgatgtgaccgaacatttggaaggccttggagtcaatcatcttaccaatttcaatggtacagttaacgaaaccgacgtttgggaagatgaaagcaaaccaaccacaagcaaatttcaatggagctctggtgaagaaaccagccaagaaagaaaccatggccaaacagtaacaccaagcagccaaaccccagatgaaaatggccatgaaagtggaaacgaaacccaagtattcagaagaattggcaccgacgaaaatgtatggtctggaacccatagcaccacgggcaatgttgatcaaagccaaaccggagaaagctggtggaccaacaaacatgaacatacctggtctgtcttgaggcttggccaaaccaacggtgaagaatctcaaaacgttgacagcaaacaacaacaagtaaacccagaaacccaaaccttggaataagataccgaagataaccatgttcttcaattggtgagctggttgagtggagttgacagcaccagcaatgacaccacagatcattggtgggaaaattggtaagatccaagctggagaagcagtttcaatggtgtaaacgtggttgttgaaaatggtgaagaaagccatgacacagtagatgaaagagacagcaacgtagatgtagtataagattctgataacccagaccatccattcaccagtgtctgggtaagcgtagatggccaacatgtcaatgaaagtggagatggatagcaaacaggtagcaatgaataatttttccaagtggtggttccaggagtccttgatggtagatgggtatttgatgaatctgaacaacatacaagaaccaaacaaagagaataagaagatttgtaagatgtagacaatcttaccaatggtgttcaaaccgtagaatctgaatgggaaagaaccaatgatcaaaccgacaccaccagtggccatggtacaagcaaaccaggaccaggtgaaatgcttcaatctttgagacaatggaacgtgtggagccttgacgttccagtccaacaattcatggtaacgttgcttcaagatttccttcaattcacccattttgttttagtgtttgtgtgttgataagcagttgcttggttttttatgaaaaatagctagaaggaataagggattacaagagagatgttacaagaaagaagtaaaataaatttgattaatattgccattatcaaaagctatttatatgttgaaatcgtggagatcatgtgtgccagaaaaggccacagtttccggggagaggcataccttgaggtggctaggaatcacggagacctcttgacttgcagggtaggctagctagaattaagtgaggtgacaaggtttccatacagttttgaccttgagacgttgctacttacgatttgcagtatgcaagtctcatgctgcaaacaaaagaggaccgctcaggtaatcgctcaattagtggacgttatcaggggcgggagaggcgaaagtggtttttggtggtgtaagtaaaggtcgtccaaatatgcaggtgtttgggtgctatcctagtggaagctcggatcagtagataacccgcctagaagcggtatttttctttttttttcttccttctttttcgtcattatttcaaacgcttttgcgtcaagtaatgaatatctggcggttccgcggggcgcgccgggcc

SEQ ID NO: 27 - DNA, 12279 bp, Artificial Sequence
  plasmid pSUC091
aagcttgcatgcctgcaggtcgacggcgcgccgggcccgtttaaacggccggccaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggacgcgtgcggccgcggatccacgctgacatggtttctttaggtttgatgaggccgtcttttgttgatagcagctttttccattttttttttttttgtttcgagtaacgtatggtttagtatctgtcttctccttctcttacaaaaaaaccctttgtaaaatagtgccgagttggaggacatcaatctgatgggcaagaaaacaccaaccccccctatatgaaaagaaaatgataagcagatagataaaaatacttaattaactaatacataaaaataagaggtatataaaaatattatatggaagcaataattattactccttacttcttttgggatggaggcaaagtttcttcttccaagacaaccaacaaatcagaagcatcgacggattcaccatctttgatgaaaacgtccttgacttgaccgtcagctggagaagagacaaccatttccatcttcatagcggacaaaacagcaatagattcacccttcttgaccaaggaacccttgtggaccttgacttcgatgataacaccagccattggagcaccgatttggtgggtgtcgtgaacatcagcctttggcttggcaacggattggatgttttgagatttgtcagcaactctgatctttctcaattcaccgtttaattcgaagtagacttctctttgaccggttttcttgttcaagtcaccaacagcttgtaatttgatgatcaaagtcttaccttgttcaatggtgacttcaatttcttcatctggttcagctggggccaaaaagttcttggttggtaagacggacaaatcaccgtaggtttctctgatcttttggaaatcttcgtaaactcttgggtacatgttgtaagaagcaacatcacattcgtcaatatcaccgaatctgttttgcaaatcttctctgatcttttccaagtcaaatggttccaattccaaacctggtctacaagtcaactttcttctcttgtttcttaagacgtcagaacgtaatggttctgggaaaccaccgtatggttgaccgatcaaaccttcaaagaagtccatgacggaatctgggaagtctagagagttagccaatcttctgatgtcatcagaggttaatttgttgctgaccatgaattgggccaagtcaccaacaaccttggaggttggagtaaccttgacaatgtcacccaacaagtagttggcttctctgtaagctctcttggtttcagcccattgttcacctaaacccaattgttgagcttggaacaataagttggtcaattgaccacctggaatttcgtgttggtagacttctgggtctggacccttcaagtcagcttcgaaacaagagtacaataatctcatttcagcccagtaagcatctaattctctaacgtgttcgacgttgataccggtgtcgatgttaccttccaaggaggccaacaaagcgttaatggatggttgagaggtcaaaccagacatggagttgatggcaacatcgacgacatcagcaccagccaaagcacaagcagtcatggaagcaacggcagtaccggcagaatcgtgagagtggacgtgaattggcaaatctgggtatctggttctcaaagaaccgatcaacaacttagcagcagctggcttcatggtaccagccatgtccttgatacctaagatgtgggtacccatttggacgatcttttcaacaacttccaagtagtagtccaagttgtacttcttaccaggttgcaacatgtcaccggagtaacaaacggtagcttcaacaacaccaccagccttcttgacagcgttaacaccgacctttaattgttccaagtcattcaaagcatcgaaaactctgaagatgtcaacaccgttgtccttagcttgcttgacgaagtggtcaatggcattgtctggtaaagaagagtaagcaacaccgttagcacctcttagcaacatttggaatggaatgtttggaaccaaagatctcaactttcttagtctttcccatgggtcctcgtgcaagaatctcatggcgacatcgaaggtggcaccaccccaacattccaaggcgaaagcaccggccaaggcatgagcggtggttggagcgatagtagccaaatcgtgggttctaactctggtagccaataaagattggtgagcatctctccaggtggtgtccatcaacaaagtaccgttgaattgtctgacttgcttggcaaattcagatggacccttttccaacaaaacttgtctccaaccagatggtggagcagatttagtaacgttgatgacgttaccttgagcgtcgtgcaagtgtggaacggaagggttagatttcaactttggtaaaccaatttgacccttgatagaagaaccgttgacagccaaatcagccaagtagtgcaacaatttttgagctctgttttgagaggaaaccatttggaacaattgtggagtgtcgtcaatgaaagtggtccagtaagtaccttcaatgaaaacagggttggtcaacaaagtcaacaaaaatggaatgttggtcttaacacctctgattctgaattcgatcaaagctctgatcatctttcttctgacaatttcgtaagtggaaccggaacaggaacatttgaccaacatggagtcgtagtgaggagagatagtagcaccagcgtaagcgttaccaccatccaatctgacaccgttaccaccagcagatctgtaaacttccaaacgaccggtgtctggttggaagttcttggatgggtcctcggtagtgatacgacattggatggagaaacctctagtggtgatcttgtcttgcaacaaacccaattgagtcaaagtggcaccagcagcaatttggatttgagcagagacaatgtcaataccagtgatttcttcagtgatagtgtgttcgacttgaattcttgggttaatttcgatgaaataatgtctgttttggttgtcaaccaagaattcagcagtaccagcgtttctgtaaccacaaaccttagctaatttgacagcgtcagtcaagatagcatcacggacttctcttggtaaagtcttggctggagcaacttcaacaaccttttggtgacgacgttgaacagaacagtctctttcgaacaaatggacaacgttaccgtggttgtcagccaacaattgaacttcaatgtgctttggcttgtccaagaatctttcgacgaaacaggtaccgttaccgaaagcagttctggcttcagaggtagctctttggaaagcatcagcaacgtcatcaccttctctaacaactctcataccacgaccaccaccaccgaaagcggccttgatgataactgggtaaccgtattcattgacgaaatccaaggcttcttgaacagtttcaattggacctggagtacctggaacagttgggacattggcacgagcagccaaatgacgagcagagactttgtcaccgacagagtcaatgacttcagctggtggaccgatccaagtgataccagccttgacaaccttgtcagcaaattcagagttttcggacaagaaaccgtaacctgggtggatgaaatcgaccttgtgcttcttggcaatttcaataatttcgtccatggccaagtaggcaccaactggagtgtattgaccttcttcaccaataacgtaagcctcatcagccttcaatctgtgcatggacaaacgatcttcatgagagtagatagcaatggttctcatggatagttcatgagcagatctgaaaattctgattgggatttcacctctgttagcaaccaagattttgttcttttcacccaacaaagagaagttgtctctcaaaccggccaatttcttggaagaggacatgtttagttaattatagttcgttgaccgtatattctaaaaacaagtactccttaaaaaaaaaccttgaagggaataaacaagtagaatagatagagagaaaaatagaaaatgcaagagaatttatatattagaaagagagaaagaaaaatggaaaaaaaaaaataggaaaagccagaaatagcactagaaggagcgacaccagaaaagaaggtgatggaaccaatttagctatatatagttaactaccggctcgatcatctctgcctccagcatagtcgaagaagaattttttttttcttgaggcttctgtcagcaactcgtattttttctttcttttttggtgagcctaaaaagttcccacgttctcttgtacgacgccgtcacaaacaaccttatgggtaatttgtcgcggtctgggtgtataaatgtgtgggtgcaacatgaatgtacggaggtagtttgctgattggcggtctatagataccttggttatggcgccctcacagccggcaggggaagcgcctacgcttgacatctactatatgtaagtatacggccccatatatatatatatatatatacattaaacattattggtaagggcccggccggccaagctttagagctcatggcgcgcctaggtaaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttccacacggatccacagcctagccttcagttgggctctatcttcatcgtcattcattgcatctactagccccttacctgagcttcaagacgttatatcgcttttatgtatcatgatcttatcttgagatatgaatacataaatatatttactcaagtgtatacgtgcatgctttttttacggtttaaacatttaaatgggccgctctagaggatccccgggtaccgagctcgggcccagcgctactagttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgagacctctgacatgcttttgacaggaatagacattttcagaatgttatccatatgcctttcgggtttttttccttccttttccatcatgaaaaatctctcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagctagttaaagaaaatcattgaaaagaataagaagataagaaagatttaattatcaaacaatatcaatatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttaacaaactgcgcccacgagttcttccctgacgccgctctcgcgcaggcaagggaactcgatgaatactacgcaaagcacaagagacccgttggtccactccatggcctccccatctctctcaaagaccagcttcgagtcaagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgaggacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccataggtcgagaatttatacttagataagtatgtacttacaggtatatttctatgagatactgatgtatacatgcatgataatatttaaacggttattagtgccgattgtcttgtgcgataatgacgttcctatcaaagcaatacacttaccacctattacatgggccaagaaaatattttcgaacttgtttagaatattagcacagagtatatgatgatatccgttagattatgcatgattcattcctacaactttttcgtagcataaggattaattacttggatgccaataaaaaaaaaaaacatcgagaaaatttcagcatgctcagaaacaattgcagtgtatcaaagtaaaaaaaagattttcgctacatgttccttttgaagaaagaaaatcatggaacattagatttacaaaaatttaaccaccgctgattaacgattagaccgttaagcgcacaacaggttattagtacagagaaagcattctgtggtgttgccccggactttcttttgcgacataggtaaatcgaataccatcatactatcttttccaatgactccctaaagaaagactcttcttcgatgttgtatacgttggagcatagggcaagaattgtggcttgagatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

(19) United States
(12) Patent Application Publication
(10) Pub. No.: US 2013/0171704 A1
(43) Pub. Date: Jul. 4, 2013

(54) DICARBOXYLIC ACID PRODUCTION PROCESS

(71) Applicants: Mickel Leonardus August Jansen, Den Haag (NL); Laurent Segueilha, Saint Andre-lez-lille (FR); Rene Verwaal, Nootdorp (NL); Melanie Louchart, Merville (FR)

(72) Inventors: Mickel Leonardus August Jansen, Den Haag (NL); Laurent Segueilha, Saint Andre-lez-lille (FR); Rene Verwaal, Nootdorp (NL); Melanie Louchart, Merville (FR)

(73) Assignees: ROQUETTE FRERES SA, Lestrem (FR); DSM IP ASSETS B.V., Heerlen (NL)

(21) Appl. No.: 13/821,125

(22) PCT Filed: Sep. 20, 2011
(86) PCT No.: PCT/EP11/66267

§ 371 Date: Mar. 6, 2013

Related U.S. Application Data
(60) Provisional application No. 61/386,058, filed on Sep. 24, 2010.

(30) Foreign Application Priority Data
Sep. 24, 2010 (EP) 10179450.1

(51) Int. Cl. C12P 7/44; C12P 7/62; C12P 7/46

(52) U.S. Cl. CPC C12P 7/44; C12P 7/46; C12P 7/62
USPC 435/135; 435/142; 435/145; 423/579; 252/372

(57) ABSTRACT

Process for producing a dicarboxylic acid comprising fermenting a microorganism in a suitable fermentation medium wherein a gas flow comprising 30% to 100% v/v oxygen as measured under atmospheric pressure is added to the fermentation medium and producing the dicarboxylic acid.

The present invention relates to a process for the fermentative production of a dicarboxylic acid

Dicarboxylic acids, such as malic acid, fumaric acid and succinic acid, are important compounds which are used in the food industry for the preparation and preservation of food, in the medical industry for the formulation of medical products and for other industrial uses, such as monomers for (bio) polymers. Dicarboxylic acids can be produced by petrochemical processes or fermentation based processes, by either bacteria or fungal cells.

Bacteria that have been studied for dicarboxylic acid production, such as succinic acid, are for example E. coli, Mannheimia sp., Actinobacillus sp. or Corynebacteria.

Suitable fungal cells for the production of dicarboxylic acid are for instance yeast, such as Saccharomyces or Yarrowia species, or filamentous fungi such Aspergillus or Rhizopus species.

Several processes for the production of dicarboxylic acids were developed.

WO2009/081012 discloses a process for the production of succinic acid by fermenting an Escherichia coli strain under anaerobic conditions and high carbon dioxide concentration.

WO2008/14462 discloses a process for the production of malic acid and succinic acid by fermenting a yeast under different carbon dioxide concentrations.

The aim of the present invention is an alternative process for the production of a dicarboxylic acid at a sufficiently high yield.

### SUMMARY OF THE PRESENT INVENTION

The present invention relates to a process for producing a dicarboxylic acid comprising fermenting a microorganism in a suitable fermentation medium wherein a gas flow comprising 30% to 100% v/v oxygen as measured under atmospheric pressure is added to the fermentation medium and producing the dicarboxylic acid.

The present disclosure also relates to the use of a gas flow comprising 30% to 100% v/v oxygen for producing a dicarboxylic acid by a microorganism in a suitable fermentation medium

### DEFINITIONS

The terms “dicarboxylic acid” and “dicarboxylate”, such as “succinic acid” and “succinate” have the same meaning herein and are used interchangeably, the first being the hydrogenated form of the latter.

The term fermenting or fermentation as used herein refers to the microbial production of compounds such as alcohols or acids from carbohydrates.

A genetically modified or recombinant microorganism, or genetically modified or recombinant microbial cell according to the present disclosure is defined herein as a cell which contains a disruption of a gene or contains, or is transformed or genetically modified with a nucleotide sequence that does not naturally occur in the microbial cell, or it contains additional copy or copies of an endogenous nucleic acid sequence. A wild-type microbial cell is herein defined as the parent cell of the recombinant cell.

Disruption, or deletion or knock-out of a gene means that part of a gene or the entire gene has been removed from a cell, or a gene has been modified such that the gene is not transcribed into the original encoding protein.

The term “homologous” when used to indicate the relation between a given (recombinant) nucleic acid (DNA or RNA), gene or polypeptide molecule and a given host organism or host cell, is understood to mean that in nature the nucleic acid or polypeptide molecule is produced by a host cell or organisms of the same species, preferably of the same variety or strain.

The term “heterologous” when used with respect to a nucleic acid (DNA or RNA) or protein refers to a nucleic acid, gene or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but have been obtained from another cell or synthetically or recombinantly produced.

A gene in a recombinant microorganism as disclosed herein may be overexpressed. There are known methods in the art for overexpression of genes encoding enzymes. A gene encoding an enzyme may be overexpressed by increasing the copy number of the gene coding for the enzyme in the cell, e.g. by integrating additional copies of the gene in the cell's genome, by expressing the gene from a centromeric vector, from an episomal multicopy expression vector or by introducing an (episomal) expression vector that comprises multiple copies of one or more gene(s).

Overexpression of a gene encoding an enzyme according to the invention may also be achieved with a (strong) constitutive promoter.

Promoters for microbial cells, such as bacteria and fungi are generally known to the skilled person in the art. Suitable promoters for fungal cells may be, but are not limited to, TDH1, TDH3, GAL7, GAL10, GAL1, CYC1, HIS3, ADH1, PH05, ADC1, ACT1, TRP1, URA3, LEU2, ENO1, TPI1, AOX1, PGL, GPDA and GAPDH. Other suitable promoters include PDC1, GPD1, PGK1, and TEF1.

A gene encoding an enzyme may be ligated into a nucleic acid construct, for instance a plasmid, such as a low copy plasmid or a high copy plasmid. The microbial cell according to the present invention may comprise a single copy, but preferably comprises multiple copies of a gene, for instance by multiple copies of a nucleotide is construct.

A nucleic acid construct may be maintained episomally and thus comprises a sequence for autonomous replication, such as an autonomously replicating sequence and a centromere (Sikorski and Hieter, 1989, Genetics 122, 19-27). In the event a microbial cell in the process of the present invention is a fungal cell, a suitable episomal nucleic acid construct may e.g. be based on the yeast 2μ or pKD1 plasmids (Gleer et al., 1991, Biotechnology 9: 968-975), or the AMA plasmids (Fierro et al., 1995, Curr. Genet. 29:482-489). Alternatively, each nucleic acid construct may be integrated in one or more copies into the genome of the microbial cell. Integration into the cell's genome may occur at random by non-homologous recombination. The nucleic acid construct may also be integrated into the cell's genome by homologous recombination as is well known in the art.

### DETAILED DESCRIPTION

The present invention relates to a process for producing a dicarboxylic acid as comprising fermenting a microorganism in a suitable fermentation medium wherein a gas flow comprising 30% to 100% v/v oxygen as measured under atmospheric pressure is added to the fermentation medium and producing the dicarboxylic acid. In one embodiment, a gas flow that is added to the fermentation medium comprises for example 40% to 100% v/v, or 50% to 100% v/v for example 70% to 100% v/v or 80% to 100% v/v or 90% to 100% v/v oxygen, or 95% to 100% v/v oxygen, or about 100% v/v oxygen as measured under atmospheric pressure.

A skilled person understands that a local pressure in a fermenter may vary. The local pressure in a fermenter is usually the result of hydrostatic pressure and the pressure in the headspace of a fermenter. The pressure in the headspace may be atmospheric pressure. Usually a slight overpressure is applied in the headspace, for example between 0.1 and 0.5 bar overpressure. Overpressure as used herein, is any pressure higher than atmospheric pressure.

We surprisingly found that adding a gas flow of 30% to 100% v/v oxygen (O₂), to the fermentation medium resulted in a high yield (product per substrate) of dicarboxylic acid such as succinic acid, which was comparable to a yield when a gas flow comprising excess of carbon dioxide (CO₂) was used. A high yield as used herein is defined as an amount of dicarboxylic acid/substrate of at least 0.3, for instance at least 0.35, for instance at least 0.4, 0.5, 0.6, 0.7 or 0.8 and usually below a yield of 1.

The process according to the present invention was found advantageous for the production of dicarboxylic acid in which one or more carbon dioxide molecules are incorporated during fermentation. Surprisingly, it was found that a microorganism as disclosed herein was able to respirate sufficient oxygen under high oxygen pressure into carbon dioxide, which was converted into a dicarboxylic acid.

The process according to the present invention was found in particular advantageous for microorganisms which are not able to derive sufficient energy (ATP) from the fermentative production of a dicarboxylic acid

Another advantage of the process according to the present disclosure is that there is no need for an additional carbon dioxide gas flow to produce a dicarboxylic acid at a sufficiently high yield. This was found in particular advantageous for a process for producing a dicarboxylic acid at an industrial scale.

In one embodiment, the process according to the present invention is carried out at a partial pressure of oxygen (pO₂) ranging between 0% and 10%, for instance between 0.1% and 8%, for instance between 0.5% and 5%, or between 1% and 2%. It was found that by keeping a partial pressure of oxygen below 10%, oxygen accumulation could be prevented and resulting toxic conditions for the microorganism to occur. A partial pressure of oxygen (O₂) ranging between 10% and 0% may for instance be maintained by stirring the fermentation medium and/or by sparging a gas flow through a fermentation medium. Another advantage of carrying out a process for producing a dicarboxylic acid at a partial pressure of oxygen below 10%, was that a higher yield of dicarboxylic acid can be obtained as compared to a process carried out at a partial pressure of oxygen above 10%.

A dicarboxylic acid that may be produced by a process as disclosed herein may for instance be succinic acid, fumaric acid, malic acid or adipic acid, for instance succinic acid.

Adding a gas flow comprising 30% to 100% v/v oxygen to a fermentation medium in a process of the present disclosure, may be carried out in any suitable way, for instance by adding the gas flow to a liquid phase or gas phase in a fermenter, which is known to the skilled person in the art. Adding a gas flow comprising 30% to 100% v/v oxygen is preferably carried out in a continuous way. Adding a gas flow comprising oxygen in a continuous way means that oxygen may be added constantly, i.e. without interruption to the fermentation medium. Alternatively, oxygen may be added continuously to the fermentation medium at short intervals of between 1 sec and 5 min, for instance between 5 sec and 2 min, for instance between 10 sec and 1 min.

A process according to the present disclosure comprises fermenting any suitable microorganism capable of producing a dicarboxylic acid, for instance a bacterial or a fungal cell. A suitable bacterial cell may for instance belong to Mannheimia, such as M. succiniciproducens, Actinobacillus, such as A. succinogenes, Anaerobiospirillum, Bacteroides, Succinimonas, Escherichia, such as E. coli. A suitable fungal cell may for instance belong to genera Saccharomyces, Aspergillus, Penicillium, Pichia, Kluyveromyces, Yarrowia, Candida, Hansenula, Humicola, Issaichenkia, Torulaspora, Trichosporon, Brettanomyces, Rhizopus, Zygosaccharomyces, Pachysolen or Yarnadazyra. A fungal cell may for instance belong to a species of Saccharoryces cerevisiae, Saccharomyces uvarur, Saccharomyces hayanus, Aspergillus niger Peniciliurn chrysogenum, Pichia slipidis, Kluyveromyces marxianus, K. lactis, K. thermotolerans, Yarrowia lipolytica, Candida sonorensis, C. glabrata, Hansenula polymorpha, issatchenkia orientails, Torulaspora delbrueckii, Brettanoryces bruxeliensis, Rhizopus oryzae or Zygosaccharomryces baili. In one embodiment a fungal cell in the process of the present invention is a yeast, for instance belonging to a Saccharomyces sp., such as a Saccharomyces cerevisiae.

In one embodiment the process as disclosed herein comprises fermenting a microorganism that is genetically modified, i.e. a recombinant microorganism. A recombinant microorganism may for instance be a recombinant yeast, for instance a recombinant Saccharomyces, such S. cerevisiae.

A recombinant microorganism as used herein, may express, for instance, may overexpress a gene encoding a phosphoenolpyruvate (PEP) carboxykinase. Any PEP-carboxykinase catalyzing the reaction from phosphoenolpyruvate to oxaloacetate (4.1.1.49) may be suitable for overexpression in a microbial cell. A microorganism may express a heterologous PEP carboxykinase, such as a PEP carboxykinase derived from Escherichia coli, Mannheimia sp., Actinohacillus sp., or Anaerobiospirilium sp., more preferably Mannheiria succiniciproducens, Actinobacillus succinogenes, or Anaerobiospirilium succiniciproducens.

A microorganisms in the process as disclosed herein may express, for instance may overexpress, a nucleotide sequence encoding a pyruvate carboxylase (PYC), for instance an endogenous or homologous pyruvate carboxylase may be overexpressed.

A microorganism as used herein may further express, for instance may overexpress, a gene encoding a malate dehydrogenase (MDH). A MDH may be any suitable homologous or heterologous malate dehydrogenase, catalyzing the reaction is from oxaloacetate to malate (EC 1.1.1.37). In the event a microorganism is a yeast such as S. cerevisiae, a MDH may be MDH3 from S. cerevisiae.

A microbial cell as used herein may express, for instance may overexpress a gene encoding a fumarase, that catalyses the reaction from malic acid to fumaric acid (EC 4.2.1.2). A gene encoding fumarase may be derived from any suitable origin, for instance from microbial origin, for instance from a yeast such as Saccharornyces or a filamentous fungus, such Rhizopus oryzae.

A microorganism as used herein may express, for instance may overexpress any suitable heterologous or homologous gene encoding a NAD(H)-dependent fumarate reductase, catalyzing the reaction from fumarate to succinate (EC 1.3.1.6). The NADH-dependent fumarate reductase may be a heterologous enzyme, which may be derived from any suitable origin, for instance bacteria, fungi, protozoa or plants. A microorganism as used herein may comprise a heterologous NAD(H)-dependent fumarate reductase, for instance derived from a Trypanosoma sp, for instance a Trypanosoma brucei.

A microorganism may express, for instance may overexpress, a gene encoding a dicarboxylic acid transporter protein. A dicarboxylic acid transporter protein may be a homologous or heterologous protein. A dicarboxylic acid transporter protein may for instance be a malic acid transporter protein (MAE) from Schizosaccharomyces pombe.

A recombinant microorganism in the process for producing a dicarboxylic acid disclosed herein may comprise a disruption of a gene encoding an enzyme of the ethanol fermentation pathway. A gene encoding an enzyme of an ethanol fermentation pathway, may be pyruvate decarboxylase (EC 4.1.1.1), catalyzing the reaction from pyruvate to acetaldehyde, or alcohol dehydrogenase (EC 1.1.1.1), catalyzing the reaction from acetaldehyde to ethanol. A microorganism in the process as disclosed herein may comprise a disruption of one, two or more genes encoding an alcohol dehydrogenase. In the event a microorganism is a yeast, e.g. Saccharomyces cerevisiae, the S. cerevisiae may comprise a disruption of an alcohol dehydrogenase gene ADH1 and/or ADH2.

A process for producing a dicarboxylic acid as disclosed herein may comprise fermenting a recombinant microorganism which overexpresses a gene encoding an enzyme selected from the group consisting of a phosphoenolpyruvate carboxykinase, malate dehydrogenase, a fumarase, a NAD(H)-dependent fumarate reductase, a pyruvate carboxylase and a dicarboxylic acid transporter protein. Preferably, a recombinant microorganism is a fungal cell, such as yeast, for instance Saccharomyces, for instance S. cerevisiae. Said genes may for instance be integrated into the cell's genome.

In the event a microorganism in a process of the present disclosure is a fungal cell, such as a yeast, genes as described herein are preferably expressed in the cytosol. Cytosolic expression may for instance be obtained be removal of a mitochondrial or peroxisomal targeting signal in the event such targeting signals are present in the genes encoding suitable enzymes for producing a dicarboxylic acid according to the present invention.

The fermentation medium in the process of the present invention may comprise any suitable nutrients allowing production of a dicarboxylic acid by fermenting a microorganism, such a carbon source, nitrogen source and trace elements. A suitable carbon source may for instance be glucose, fructose, galactose, xylose, arabinose, sucrose, lactose, maltose, raffinose or glycerol. A suitable nitrogen source may for instance be ammonium or urea.

The process for the production of a dicarboxylic acid of the present disclosure may be carried out at any suitable pH between 1 and 8. A suitable pH depends on the microorganism in the process of the present invention, which is usually known by the skilled person in the art. In the event a microorganism is a fungal cell a process according to the present invention may for instance be carried out at a pH between 2 and 7, for instance between 3 and 5.

A process for producing a dicarboxylic acid according to the present disclosure may be carried out at any suitable temperature, depending on the microorganism. A process of the present disclosure may be carried out between 5° C. and 60° C., or between 10° C. and 50° C., for instance between 15° C. and 45° C., or between 20° C. and 40° C. The skilled man in the art knows the optimal temperatures for fermenting a specific microorganism.

In another embodiment a process of the present disclosure further comprises recovering a dicarboxylic acid from the fermentation medium by a suitable method known in the art, for instance by crystallisation, ammonium precipitation or ion exchange technology.

In another embodiment, a process according to the present disclosure further comprises using a dicarboxylic acid that is prepared in a pharmaceutical, cosmetic, food, or feed product. A dicarboxylic produced in a process according to the present invention may for instance be converted into a polyester polymer. Succinic acid may for instance be further converted into polybutylene succinate (PBS).

In another embodiment the process according to present invention is carried out on an industrial scale. Industrial scale is herein defined as a fermentation process that is carried out in a volume of at least 10 liters, preferably at least 100 liters, preferably at least 1 cubic metre (m), more preferably at least 10, 100, or 1000 or 2000 cubic metres (m³), usually below 10,000 cubic metres (m³).

In another embodiment the present invention relates to the use of a gas flow comprising 30% to 100% v/v oxygen for producing a dicarboxylic acid by a microorganism in a suitable fermentation medium.

### FIGURES

FIG. 1. Physical map of plasmid pPWT006.

FIG. 2. Physical map of plasmid pPSUC044.

FIG. 3. Physical map of plasmid pPWT007.

FIG. 4. Physical map of plasmid pSUC047.

FIG. 5. Physical map of pBOL034.

FIG. 6. Physical map of pSUC091.

FIG. 7. Southern blot autoradiogram. Chromosomal DNA of wild-type strain SUC-347 was digested with PspOMI/Afel (lane 1) and BmgBIi/Afil (lane 2). The blot was hybridized with a specific MDH3-probe. Marker (M) represents a labelled 1 kB plus ladder (Invitrogen).

FIG. 8. Southern blot autoradiogram. Chromosomal DNA of wild-type strain SUC-347 was digested with NotI, SpeI and XhoI (lane 1) and ApaI (lane 2). The blot was hybridized with a specific FRDg-probe. Marker (M) represents a labelled 1 kB plus ladder (Invitrogen).

FIG. 9. Physical maps of the wild-type SIT4-locus (panel A) and after introduction of the MDH3, FUMR and SpMAE1 synthetic construct by integration of plasmid pSUC047, followed by intramolecular recombination leading to the loss of vector and selectable marker sequences (panel B). The hybridization of the probe for Southern blot and primers for diagnostic PCR are indicated.

FIG. 10. Physical maps of the wild-type SiT2-locus (panel A) and after introduction of the PCKa and FRDg synthetic construct by integration of plasmid pSUC044, followed by intramolecular recombination leading to the loss of vector and selectable marker sequences (panel B). The hybridization of the probe for Southern blot and primers for diagnostic PCR are indicated.

FIG. 11. Physical maps of the wild-type ADH1-locus and surrounding loci (panel A) and after introduction of the URA3 PCR fragment and PYC2 synthetic construct by integration of plasmid pSUC091 into strain SUC-347, resulting in strain SUC-401 (panel B). Primer binding sites for diagnostic PCR are indicated (panel B). Correct integration gives a 1356 bp band with primers SEQ ID NO: 19 and SEQ ID NO: 20 (lane 1) and a 1252 bp band with primers SEQ ID NO: 21 and SEQ ID NO: 22 (lane 2). No PCR product is expected if no integration has taken place.

### EXAMPLES

### Example 1

### Construction of Strain SUC-401: Introduction of the Genes PCKa, MDH3, FUMR, SFRDg, SpMAE1 and PYC2 into the Genome of S. Cerevisiae

1.1 Construction of an Expression Vector Containing the Genes of the Reductive TCA Cycle for Production of Succinic Acid

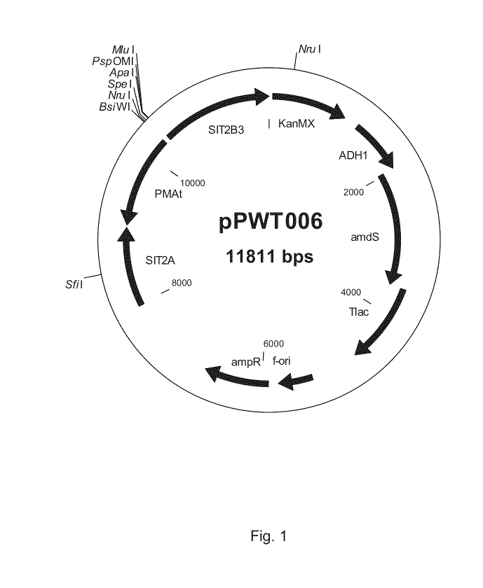

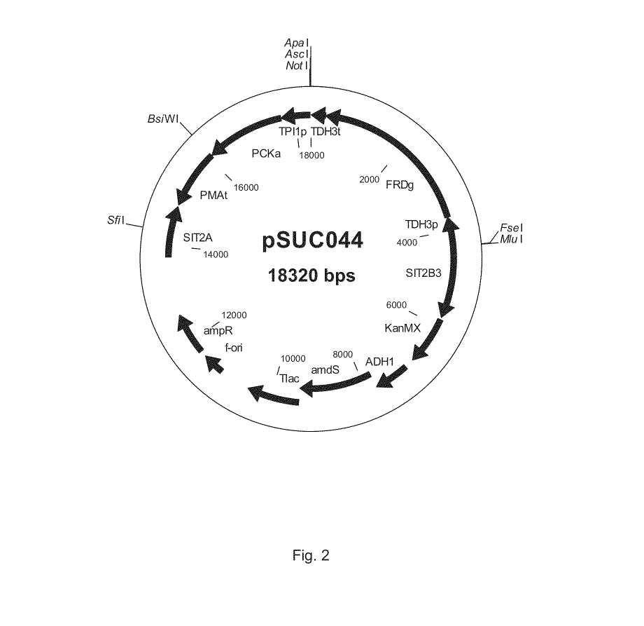

Plasmid pSUC044, as set out in FIG. 2, was constructed as follows: Plasmid pPWT006 (FIG. 1), consisting of a YGR059w (SPR3) or SIT2-locus (Gottlin-Ninfa and Kaback (1986) Molecular and Cell Biology vol. 6, no. 6, 2185-2197) and the markers allowing for selection of transformants on the antibiotic G418 and the ability to grow on acetamide, was digested with the restriction enzymes Mlul and ApaI. The kanMX-marker, conferring resistance to G418, was isolated from p427TEF (Dualsystems Biotech) and a fragment containing the amdS-marker has been described in literature (Swinkels, B. W., Noordermeer, A. C. M. and Renniers, A.C.H.M (1995). The use of the amdS cDNA of Aspergillus nidulans as a dominant, bidirectional selectable marker for yeast transformation. Yeast Volume 11, Issue 1995A, page S579; and U.S. Pat. No. 6,051,431).

The genes encoding fumarate reductase (FRDg) from Trypanosoma brucei, as disclosed in patent application WO2009/065778, and phosphoenolpyruvate carboxykinase (PCKa) from Actinobacillus succinogenes, as disclosed in patent application WO2009/065780, were synthesized by Sloning (Puchheim, Germany). Specific promoter; gene (; terminator) sequences, including appropriate restriction sites, were synthesized. The gene sequences were codon pair optimized for expression in Saccharomyces cerevisiae as disclosed in patent application WO2008/000632. The synthetic genes are under control of (or operable linked to) strong promoters from S. cerevisiae, i.e. the TDH3-promoter controlling the expression of the FRDg-gene, and the TPI1-promoter controlling the PCKa-gene. Proper termination is controlled by terminator sequences from S. cerevisiae, i.e. the TD-H3-terminator controlling the FRDg-gene and the PMAF-terminator, present on plasmid pPWT006, controlling the PCKa-gene. The TDiH3-promoter; FRDg-gene; TDH3-terminator sequence was surrounded by the unique restriction enzymes sites Mlul and ApaI. The TPi1-promoter, PCKa-gene sequence was surrounded by the unique restriction enzymes sites ApaI and BsNWI. Cloning of the FRDg synthetic construct into pPWT006 digested with Mlul and ApaI resulted in the intermediate plasmid pPWT006-FRDg. Cloning of the PCKa synthetic construct into pPWT006-FRDg digested with ApaI and BsWI resulted in plasmid pSUC044 (SEQ ID NO: 25, FIG. 2).

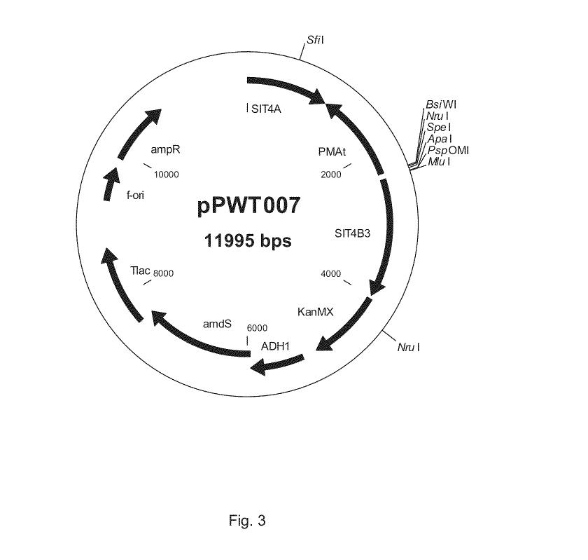

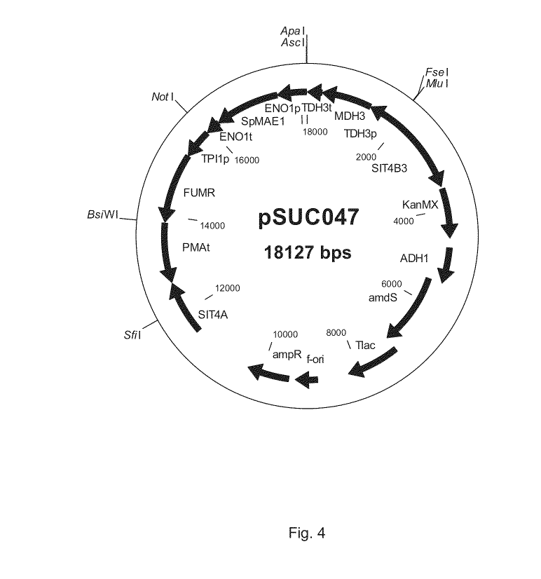

Plasmid pSUC047, as set out in FIG. 4, was constructed as follows: Plasmid pPWT007 (FIG. 3), consisting of a YEL023c or SIT4-locus (Gottlin-Ninfa and Kaback (1986) Molecular and Cell Biology vol. 6, no. 6, 2185-2197) and the markers allowing for selection of transformants on the antibiotic G418 and the ability to grow on acetamide, was digested with the restriction enzymes MluI and ApaI.

The genes encoding malate dehydrogenase (MDH3) from Saccharomyces cerevisiae, as disclosed in patent application WO2009/065778, fumarase (FUMR) from Rhizopus oryzae, as disclosed in patent application WO2009/065779, and malic acid transporter (SpMAE1) from Schizosaccharomyces pombe, as disclosed in patent application WO2009/065778, were synthesized by Sloning (Puchheim, Germany). Specific promoter; gene; terminator sequences, including appropriate restriction sites were synthesized. The gene sequences were codon pair optimized for expression in Saccharomyces cerevisiae as disclosed in patent application WO2008/000632. The synthetic genes are under control of (or operable linked to) strong promoters from S. cerevisiae, i.e. the TDH3-promoter controlling the expression of the MDH3-gene, the TP/1-promoter controlling the FUMR-gene and the ENO1-promoter controlling the SpMAE1 gene. Proper termination is controlled by terminator sequences from S. cerevisiae, i.e. the TDH3-terminator controlling the MDH3-gene, the PMA-terminator, present on plasmid pPWT006, controlling the PCKa-gene, and the ENO1-terminator controlling the SpMAE1-gene. The TDH3-promoter; MDH3-gene; TDH3-terminator sequence was surrounded by the unique restriction enzymes sites MluI and ApaI. The TPI1-promoter, FUMR-gene sequence was surrounded by the unique restriction enzymes sites ApaI. AscI and NotI at the 5′ end and BsiWI at the 3′ end. The ENO1-promoter; SpMAE1-gene; ENO1-terminator sequence was surrounded by the unique restriction enzymes sites MluI and ApaI. Cloning of the MDH3 synthetic construct into pPWT007 digested with MluI and ApaI resulted in intermediate plasmid pPWT007-MDH3. Cloning of the FUMR synthetic construct into pPWT007-MDH3 digested with ApaI and BsiWI resulted in plasmid pSUC046. Cloning of the SpMAE1 synthetic construct into pSUC046 digested with AscI and NotI resulted in plasmid pSUC047 (SEQ ID NO: 26, FIG. 4).

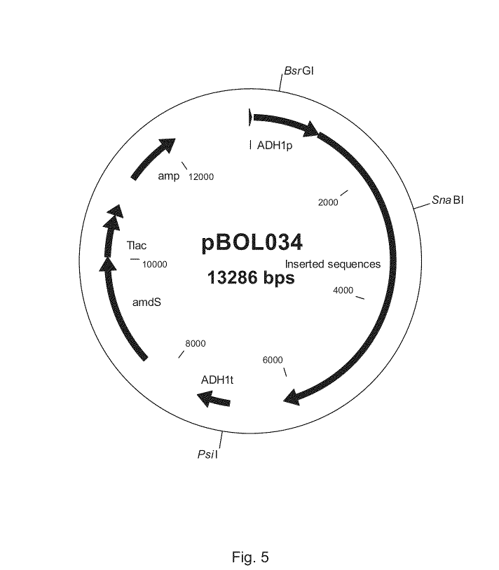

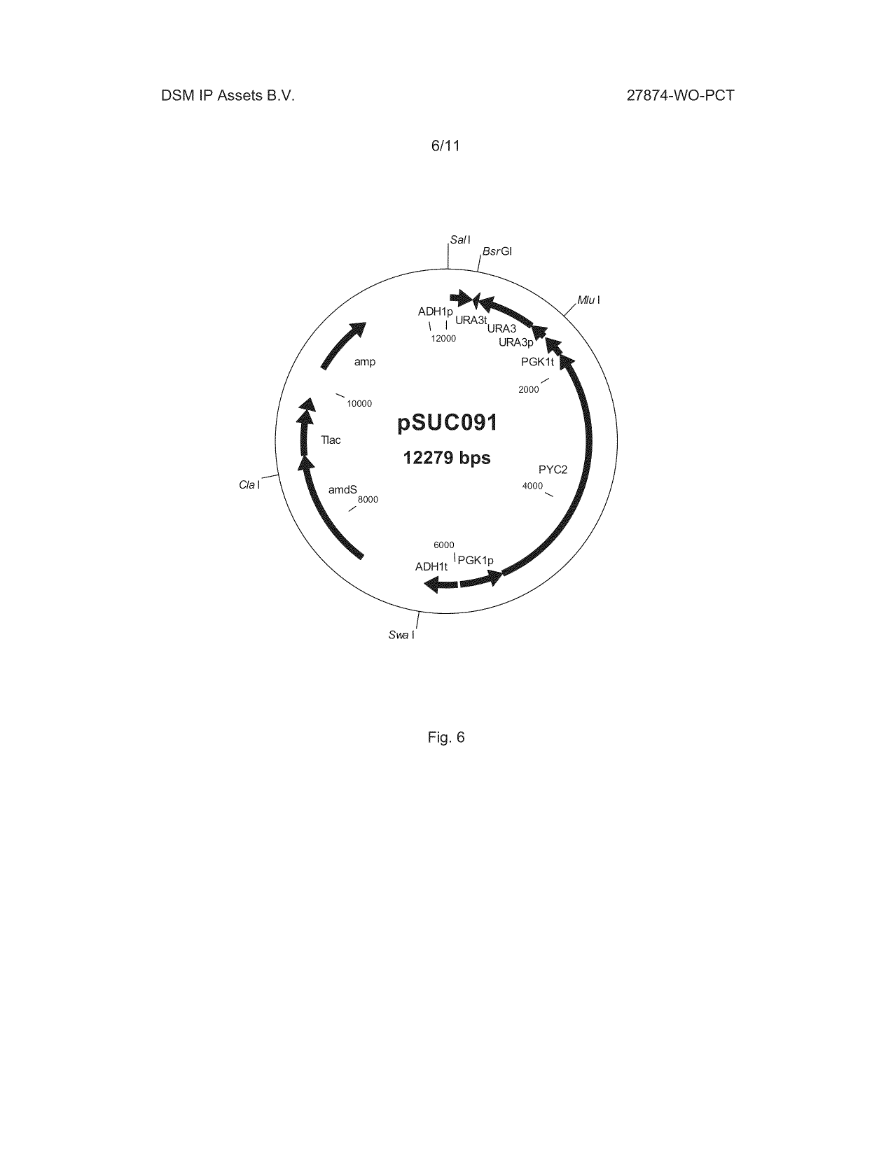

Plasmid pBOL034 (FIG. 5), consisting of a 1000 bp YOL086C (ADH1) promoter sequence (1000 bp directly upstream of start codon of YOL086C), a 500 bp YOL086C (ADH1) terminator sequence (500 bp directly downstream of stop codon) and inserted gene sequences, was used as host vector to construct pSUC091 (FIG. 6). A URA3-pomoter; URA3-gene; URA3-terminator PCR fragment (FW primer SEQ ID NO: 1, reverse primer SEQ ID NO: 2) was obtained using plasmid pRS416 (Sikorski and Hieter, 1989) as template. The primers contained appropriate restriction enzymes sites, MluI for the forward and BsrGI for the reverse primer, for further subcloning of the PCR fragment.

The gene sequence encoding pyruvate carboxylase (PYC2) from Saccharomyces cerevisiae, as disclosed in patent application WO2009/065780, was synthesized by Geneart (Regensburg, Germany). A specific promoter; gene; terminator sequence, including appropriate restriction sites was synthesized. The gene sequence was codon pair optimized for expression in Saccharomyces cerevisiae as disclosed in patent application WO02008/000632. The synthetic gene was under control of (or operably linked to) a strong promoter from S. cerevisiae, i.e. the PGK1-promoter controlling the expression of the PYC2-gene. Proper termination is controlled by a terminator sequence from S. cerevisiae, i.e. the PGK1-terminator controlling the PYC2-gene. The PGK1-promoter; PYC2-gene; PGK1-terminator sequence was surrounded by the unique restriction enzymes sites StuI and MluI. After restriction of pBOL034 with BsrGI, Psil and SnaBI, restriction of the URA3 PCR fragment with Mul and BsrGI and the PGK1-promoter, PYC2-gene, PGK1-terminator sequence with StuI and MluI, the three DNA fragments were ligated by a 3-point ligation to yield plasmid pSUC091 (SEQ ID NO: 27: FIG. 6).

1.2 Yeast Transformation

CEN.PK113-5D (MATa ura-3,52 HIS3 LEU2 TRP1 MAL2-8 SUC2) was transformed with plasmid pSUC047, which was previously linearized with SfiI (New England Biolabs), according to the instructions of the supplier. A synthetic SfiI-site was designed in the sequence of the SIT4-gene present on plasmid pPWT007 (designated SIT4A, see FIG. 3). Transformation mixtures were plated on YPD-agar (per liter: 10 grams of yeast extract, 20 grams per liter peptone, 20 grams per liter dextrose, 20 grams of agar) containing 100 μg G418 (Sigma Aldrich) per ml. After two to four days, colonies appeared on the plates, whereas the negative control (i.e. no addition of DNA in the transformation experiment) resulted in blank YPD/G418-plates. Alternatively, positive transformants were selected on agar plates containing acetamide, which can be used as a sole nitrogen source due to the presence of the acetamidase (amdS) marker after integration of the DNA construct. For this purpose, transformation mixtures were plated on agar acetamide agar plates (per liter: 20 grams of agar, 20 grams per liter potassium dihydrogen phosphate, 0.5 grams per liter of magnesiumsulfat-heptahydrat, 70 milliliters of 32% galactose, 1 milliliter of 50% dextrose, 12.5 milliliter of 400 mM acetamide (Sigma), 1 ml vitamins and 1 ml trace elements (compositions of vitamins and trace elements are described in literature (Verduyn C, Postma E, Scheffers W A, Van Dijken J P. Yeast, 1992 July; 8(7):501-517). After two to four days, colonies appeared on the plates, whereas the negative control (i.e. no addition of DNA in the transformation experiment) resulted in blank acetamide agar-plates. The integration of plasmid pSUC047 was directed to the SIT4-locus. Correct transformants with integration of the MDH3, FUMR and SpMAE1 genes at the SIT4-locus were characterized using PCR techniques. PCR reactions indicative for the correct integration at the SIT4-locus were performed with the primers indicated by SEQ ID NO: 3 and 4, and SEQ ID NO: 5 and 6. With the primer pairs of SEQ ID NO: 4 and 5, the integration of one copy of plasmid pSUC047 was checked. If plasmid pSUC047 was integrated in multiple copies (head-to-tail integration), the primer pair of SEQ ID NO: 4 and 5 will give a PCR-product. If the is latter PCR product is absent, this is indicative for one copy integration of pSUC047. Introduction of the synthetic gene sequences was confirmed by PCR for MDH3 using primers indicated by SEQ ID NO: 7 and 8, FUMR using primers indicated by SEQ ID NO: 9 and 10, and SpMAE1 using primers indicated by SEQ ID NO: 11 and 12. A strain in which one copy of plasmid pSUC047 was integrated in the Sit4-locus, designated CEN.PK113-5D-pSUC047 was used for marker rescue (see section 1.3). The resulting marker-free strain was designated SUC-270 (MATa ura3,52 HIS3 LEU2 TRP1 sit4:: TDH3p-MDH3-TDH3t; ENO1p-SpMAE1-ENO I t; TPI1p-FUMR-PMA1t MAL2-8 SUC2).

Strain SUC-270 was transformed with plasmid pSUC044, which was previously linearized with SfiI (New England Biolabs), according to the instructions of the supplier.

A synthetic SfiI-site was designed in the sequence of the SIT2-gene on plasmid pPWT006 (designated SIT2A, see FIG. 1). Transformation mixtures were plated as described above. After two to four days, colonies appeared on the plates, whereas the negative control (i.e. no addition of DNA in the transformation experiment) resulted in blank YPD/G418-plates. The integration of plasmid pSUC044 was directed to the SIT2-locus. Correct transformants with integration of the PCKa and FRDg genes at the SIT2 locus were characterized using PCR techniques. PCR reactions indicative for the correct integration at the SIT2-locus were performed with the primers indicated by SEQ ID NO: 13 and 4, and SEQ ID NO: 5 and 14. With the primer pairs of SEQ ID NO: 4 and 5, the integration of one copy of plasmid pSUC044 was checked. If plasmid pSUC044 was integrated in multiple copies (head-to-tail integration), the primer pair of SEQ ID NO: 4 and 5 will give a PCR-product. If the latter PCR product is absent, this is indicative for one copy integration of pSUC044. Introduction of the synthetic gene sequences was confirmed by PCR for PCKa using primers indicated by SEQ ID NO: 15 and 16, and FRDg using primers indicated by SEQ ID NO: 17 and 18. The resulting single copy integration strain was designated SUC-304, which was subsequently used for marker-rescue (see section 1.3), resulting in marker-free strain SUC-347 (MATa ura3,52 HIS3 LEU2 TRP1 sit2:: TPI1p-PCKa-PMA1t; TDH3p-FRDg-TDH3t sit4:: TDH3p-MDH3-TDH3t; ENO1t-SpMAE1-ENO1t; TPI1p-FUMR-PMA1t MAL2-8 SUC2). Strain SUC-347 was further analyzed by Southern blot analysis (see section 1.4).

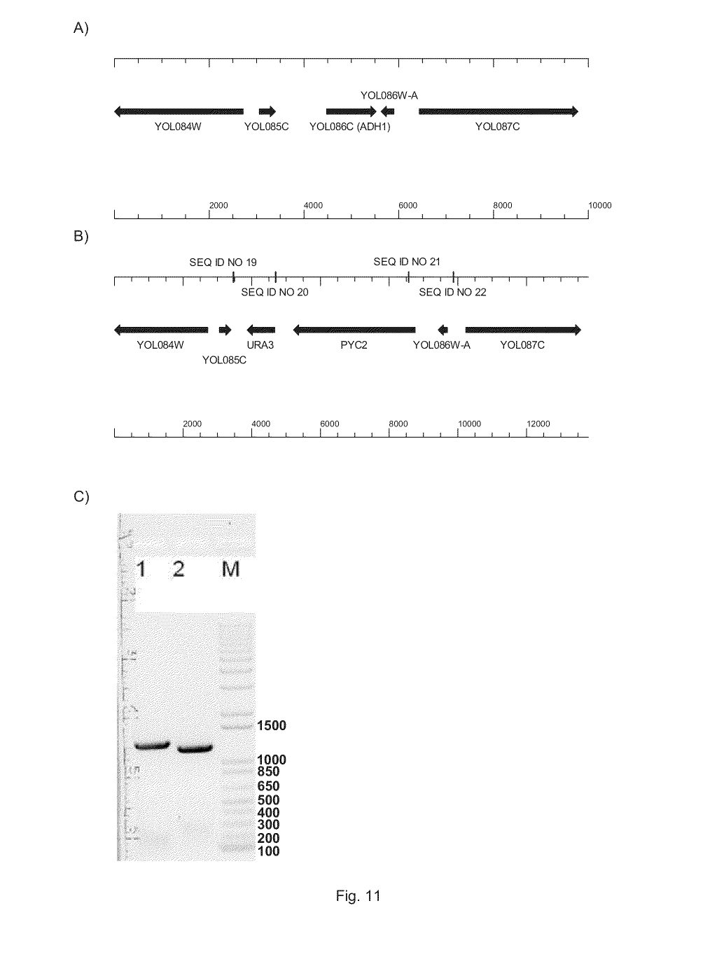

Strain SUC-347 was transformed with a 6.4 kB fragment of plasmid pSUC091, which was previously linearized with the restriction enzymes SwaI, SaiI and ClaI (FIG. 6). Transformation mixtures were plated on Yeast Nitrogen Base (YNB) w/o AA (Difco)+2% glucose. Correct transformants were initially selected for uracil prototrophy, because the parent strain had an auxotrophy for uracil (ura3,52), which was complemented by a functional copy of the URA3 gene. The transformants were further analyzed by PCR to confirm correct targeting of the URA3 PCR product and PYC2 synthetic construct into the adhil locus using primers indicated by SEQ ID NO: 19 and 20, and SEQ ID NO: 21 and 22 and presence of the PYC2 synthetic construct using primers indicated by SEQ ID NO: 23 and 24 (FIG. 11). The resulting strain was designated SUC-401 (MATa ura3,52 HIS3 LEU2 TRP1 sit2:: TPI1p-PCKa-PMA1t; TDH3p-FRDg-TDH3t sit4:: TDH3-p-MDH-3-TDH3t; ENO1p-SpMAE1-ENO1t; TP1p-FUMR-PMA1t adh1::PGK1p-PYC2-PGK1t; URA3p-URA3-URA3t MAL2-8 SUC2).

1.3 Marker rescue

In order to be able to transform the yeast strain with other constructs, using the same selection markers, it was necessary to remove the selectable markers. The design of plasmid pSUC044 and pSUC047 was such, that upon integration of pSUC044 and pSUC047 in the chromosome, homologous sequences were in close proximity of each other. This design allowed the selectable markers to be lost by spontaneous intramolecular recombination of these homologous regions.

Upon vegetative growth, intramolecular recombination will take place, although at low frequency. The frequency of this recombination depends on the length of the homology and the locus in the genome (unpublished results). Upon sequential transfer of a subfraction of the culture to fresh medium, intramolecular recombinants will accumulate in time.

To this end, strains CEN.PK113-5D-pSUC047 and SUC-304 were cultured in YPD-medium (per liter: 10 grams of yeast extract, 20 grams per liter peptone, 20 grams per liter dextrose), starting from a single colony isolate. 25 μl of an overnight culture was used to inoculate fresh YPD medium. After at least five of such serial transfers, the optical density of the culture was determined and cells were diluted to a concentration of approximately 5000 per ml. 100 μl of the cell suspension was plated on Yeast Carbon Base medium (Difco) containing 30 mM KPi (pH 6.8), 0.1% (NH4)₂SO₄, 40 mM fluoro-acetamide (Amersham) and 1.8% agar (Difco). Cells identical to cells of strains CEN.PK113-5D-pSUC047 and SUC-304, i.e. without intracellular recombination, still contained the amdS-gene. To those cells, fluoro-acetamide is toxic. These cells will not be able to grow and will not form colonies on a medium containing fluoro-acetamide. However, if intramolecular recombination has occurred, CEN.PK113-5D-pSUC047 and SUC-304 variants that have lost the selectable markers will be able to grow on the fluoro-acetamide medium, since they are unable to convert fluoro-acetamide into growth inhibiting compounds. Those cells will form colonies on this agar medium.

The obtained fluoro-acetamide resistant colonies of CEN.PK113-5D-pSUC047 were subjected to PCR analysis using primers of SEQ ID NO: 3 and 4, and 5 and 6. Primers of SEQ ID NO: 5 and 6 will give a band if recombination of the selectable markers had taken place as intended. As a result, the cassette with the genes MDH3, FUMR and SpMAE1 under control of the strong yeast promoters had been integrated in the SIT4-locus of the genome of the host strain. In that case, a PCR reaction using primers of SEQ ID NO: 3 and 4 should not result in a PCR product, since these primers bind in a region that should be lost due to recombination. If a band is obtained with the latter primers, this is indicative for the presence of the complete plasmid pSUC047 in the genome, so no recombination has taken place. If primers of SEQ ID NO: 5 and 6 do not result in a PCR product, recombination has taken place, but in such a way that the complete plasmid pSUC047 has recombined out of the genome. Not only are the selectable markers lost, but also the introduced genes. In fact, wild-type yeast has been retrieved.

The obtained fluoro-acetamide resistant colonies of SUC-304 were subjected to PCR analysis using primers of SEQ ID NO: 13 and 4, and 5 and 14. Primers of SEQ ID NO: 5 and 14 will give a band if recombination of the selectable markers had taken place as intended. As a result, the cassette with the genes PCKa and FRDg under control of the strong yeast promoters had been integrated in the SIT2-locus of the genome of the host strain. In that case, a PCR reaction using primers of SEQ ID NO: 13 and 4 should not result in a PCR product, since these primers bind in a region that should be lost due to recombination. If a band is obtained with the latter primers, this is indicative for the presence of the complete plasmid pSUC044 in the genome, so no recombination has taken place. If primers of SEQ ID NO: 5 and 14 do not result in a PCR product, recombination has taken place, but in such a way that the complete plasmid pSUC044 has recombined out of the genome. Not only are the selectable markers lost, but also the introduced genes. In fact, wild-type yeast has been retrieved.

1.4 Southern Blot

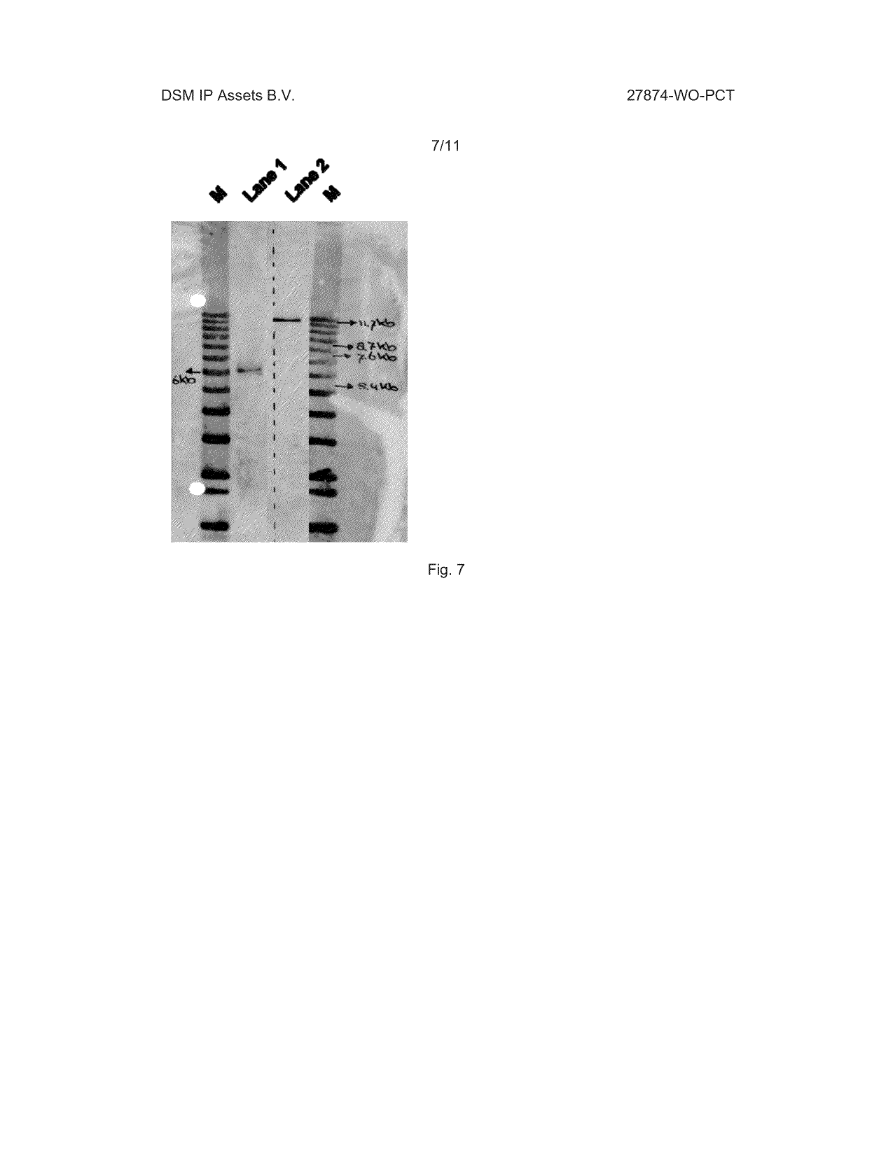

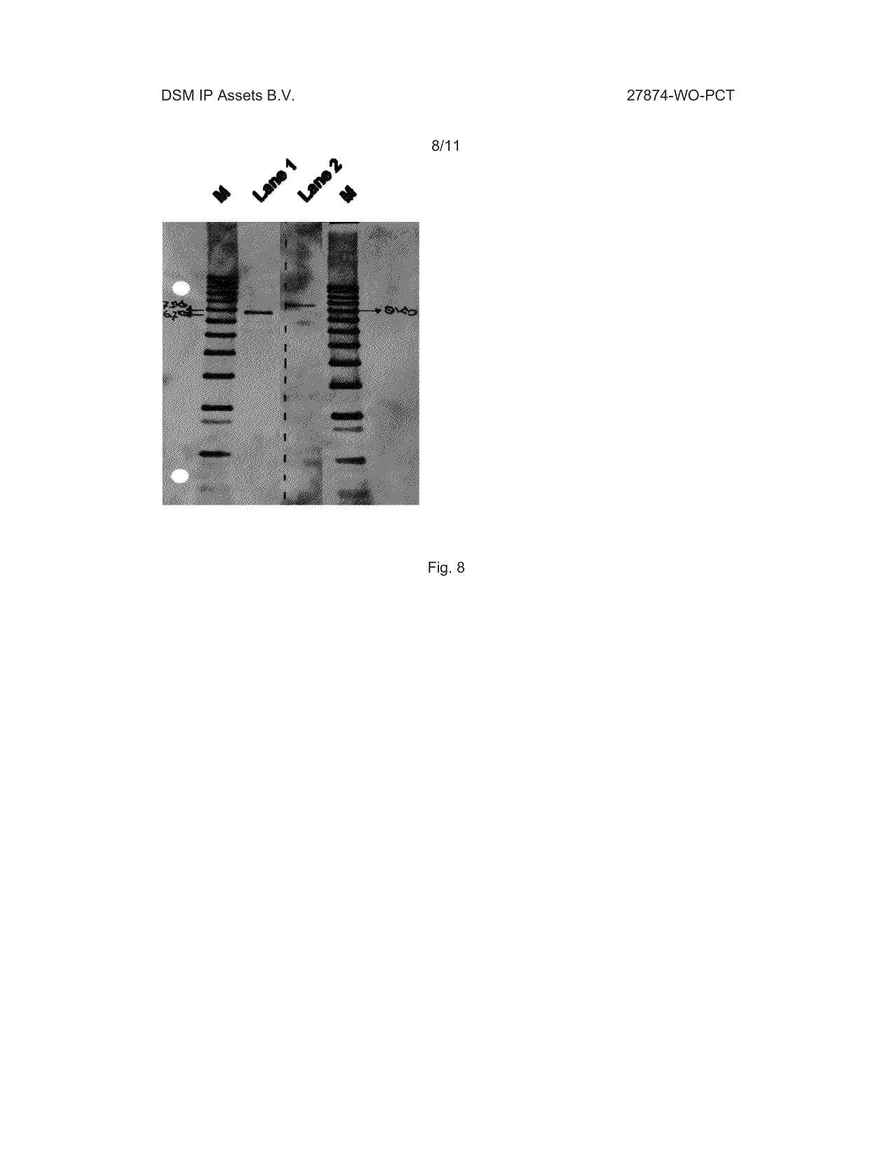

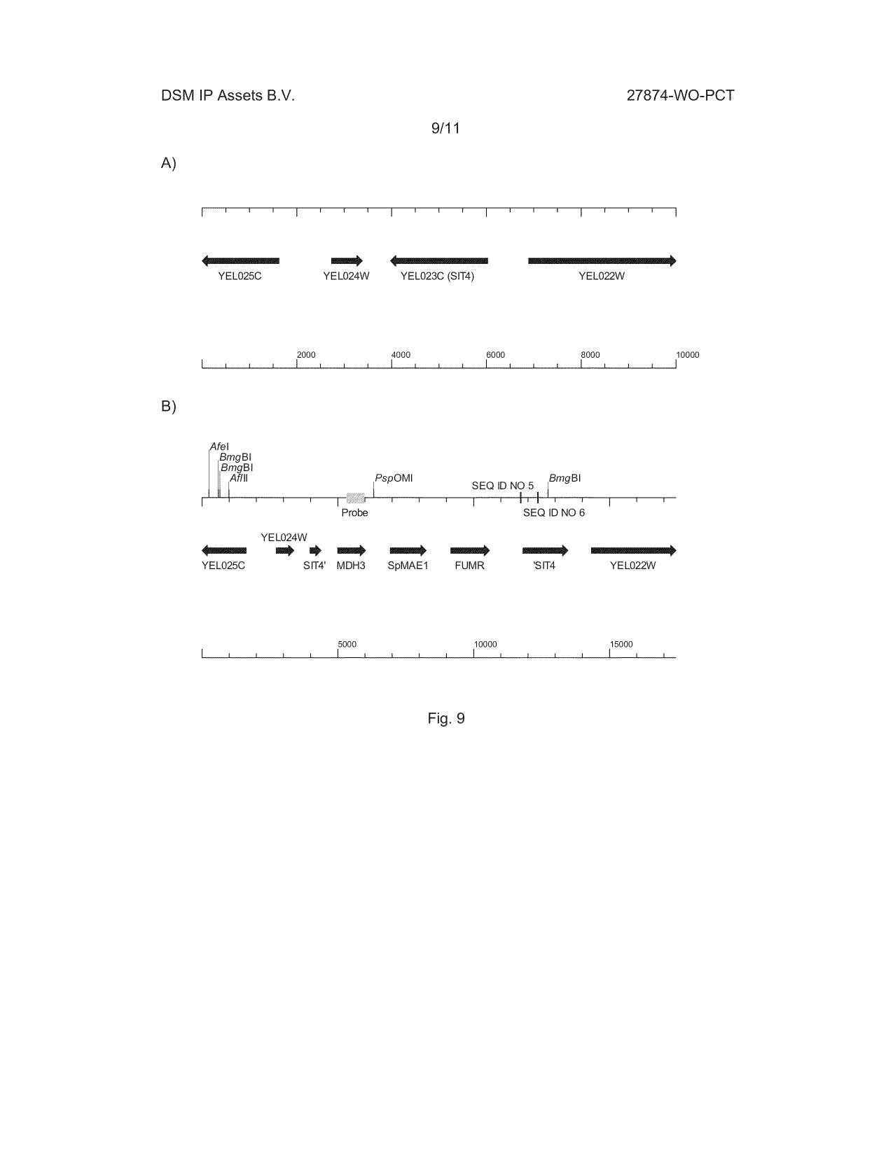

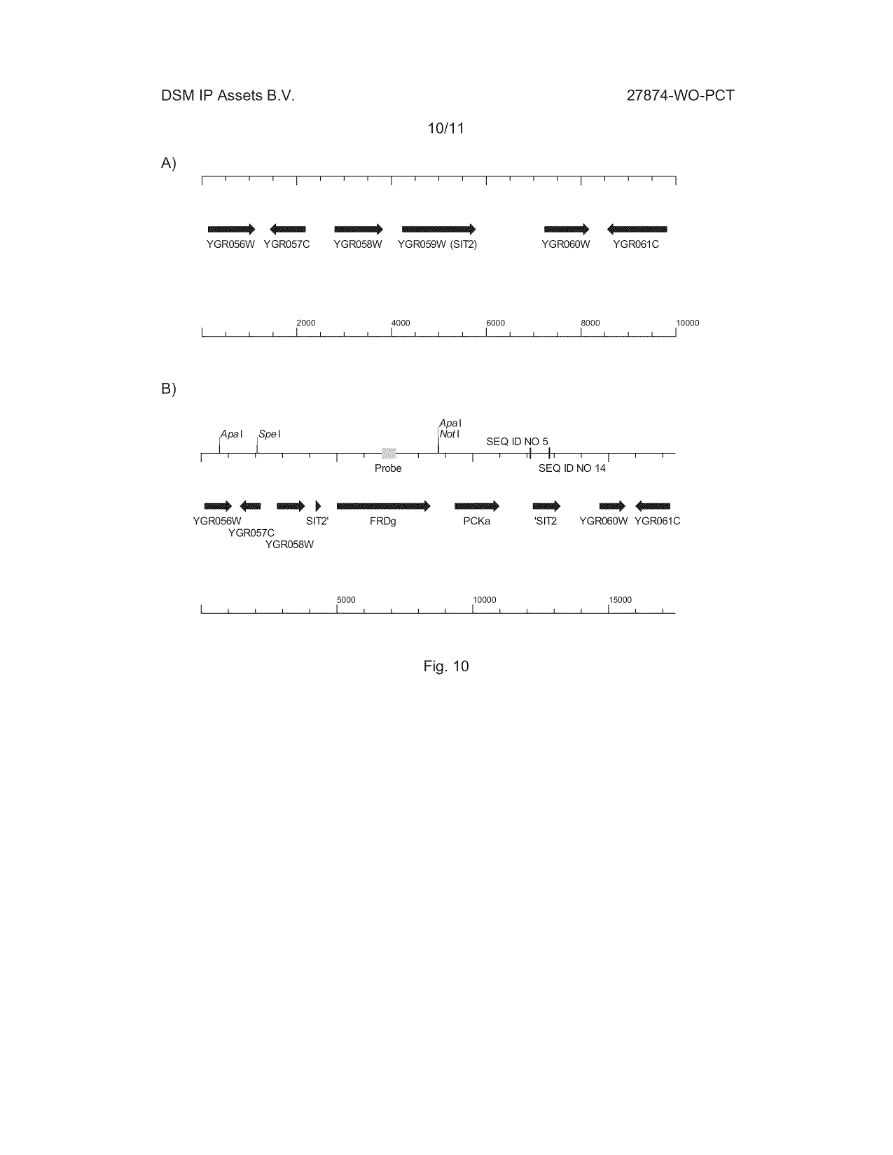

Strain SUC-347 was subjected to Southern blot analysis. Per integration locus, double checks were performed using different enzymes to restrict genomic DNA isolated from SUC-347. To confirm whether MDH3, FUMR and SpMAE1 were correctly integrated at the SIT4 locus and whether the marker sequences were out-recombined, genomic DNA from strain SUC-347 was purified and restricted with either NotI/SpeI/XhoI or ApaI. A MDH3 probe was prepared with primers of SEQ ID NO: 7 and 8, using plasmid pGBS415FUM-3 (disclosed in patent application WO2009/065778) as template. To confirm whether PCKa and FRDg were correctly integrated at the SIT2 locus and whether the marker sequence were out-recombined, genomic DNA from strain SUC-347 was purified and restricted with either PspOMIiAfel or BmgBlIAfll. An FRDg probe was prepared with primers of SEQ ID NO: 17 and 18, using plasmid pGBS414PPK-3 (disclosed in patent application WO2009/065778) as template. The results of the hybridisation experiment are shown in FIGS. 7 and 8. The expected hybridisation pattern may be deduced from the physical maps as set out in FIGS. 9 and 10 (panels B). Table 1 provides an overview of bands expected and bands observed after performing the hybridization reactions. All observed bands were as expected, indicating a marker-free strain with integration of MDH3, FUMR and SpMAE1 at the SIT4-locus and PCKa and FRDg at the SIT2-locus was obtained.

TABLE 1
Overview of Southern blot results of strain SUC-347

| | | | | Expected | Expected | |
|---|---|---|---|---|---|---|
| | | | | size if | size if | |
| Integra- | | | | not out- | out- | |
| tion | Integrated | Restriction | | recom- | recom- | Observe |
| locus | genes | enzymes | Probe | bined | bined | band |
| SIT4 | MDH3, | PspOMI/ | MDH3 | 16.7 | 6.0 | 6.0 |
| | FUMR, | AfeI | | | | |
| | SpMAE1 | | | | | |
| SIT4 | MDH3, | BmgBI/ | MDH3 | 15.0 | 11.7 | 11.7 |
| | FUMR, | AflII | | | | |
| | SpMAE1 | | | | | |
| SIT2 | PCKa, | Notl/Spel/ | FRDg | 10.7 | 7.3 | 7.3 |
| | FRDg | Xhol | | | | |
| SIT2 | PCKa, | Apal | FRDg | 20 | 8.0 | 8.0 |
| | FRDg | | | | | |

### Example 2

### Succinic Acid Production by Yeast in the Presence of Gas Flow of Different Compositions

The yeast strain SUC-401 (MATa ura3,52 HIS3 LEU2 TRP1 sit2::TPI1p-PCKa-PMA1t: TDH3p-FRDg-TDH3t sit4:: TDHp-MDH3-TDH3t:ENO1p-SpMAE1-ENO1t; TP1p-FUMR-PMAlt adh1::PGKlp-PYC2-PGK1t; URA3p-URA3-URA3t MAL2-8 SUC2) was cultivated in shake-flask (150 ml) for 3 days at 30° C. and 110 rpm. The medium was based on Verduyn medium (Verduyn C, Postma E, Scheffers W A, Van Dijken J P. Yeast, 1992 July; 8(7):501-517), but modifications in carbon and nitrogen source were made as described herein below.

TABLE 2
Preculture shake flask medium composition

| | | Concentration |
|---|---|---|
| Raw material | | (g/l) |
| Galactose | C₆H₁₂O₆•H₂O | 20.0 |
| Urea | (NH₂)₂CO | 2.3 |
| Potassium dihydrogen phosphate | KH₂PO₄ | 3.0 |
| Magnesium sulphate | MgSO₄•7H₂O | 0.5 |
| Trace element solution^{a} | | 1 |
| Vitamin solution^{b} | | 1 |

^{a}Trace elements solution

| | | Concentration |
|---|---|---|
| Component | Formula | (g/kg) |
| EDTA | C₁₀H₁₄N₂Na₂O₈•2H₂O | 15.00 |
| Zincsulphate•7H₂O | ZnSO₄•7H₂O | 4.50 |
| Manganesechloride•2H₂O | MnCl₂•2H₂O | 0.84 |
| Cobalt (II) chloride•6H₂O | CoCl₂•6H₂O | 0.30 |
| Cupper (II) sulphate•5H₂O | CuSO₄•5H₂O | 0.30 |
| Sodium molybdenum•2H₂O | Na₂MoO₄•2H₂O | 0.40 |
| Calciumchloride•2H₂O | CaCl₂•2H₂O | 4.50 |
| Ironsulphate•7H₂O | FeSO₄•7H₂O | 3.00 |
| Boric acid | H₃BO₃ | 1.00 |
| Potassium iodide | KI | 0.10 |

^{b}Vitamin solution

| | | Concentration |
|---|---|---|
| Component | Formula | (g/kg) |
| Biotin (D−) | C₁₀H₁₆N₂O₃S | 0.05 |
| Ca D(+) panthothenate | C₁₈H₃₂CaN₂O₁₀ | 1.00 |
| Nicotinic acid | C₆H₅NO₂ | 1.00 |
| Myo-inositol | C₆H₁₂O₆ | 25.00 |
| Thiamine chloride hydrochloride | C₁₂H₁₈Cl₂N₄OS•xH₂O | 1.00 |
| Pyridoxol hydrochloride | C₈H₁₂ClNO₃ | 1.00 |
| p-aminobenzoic acid | C₇H₇NO₂ | 0.20 |

Subsequently, the content of the shake-flask was transferred to a seed fermenter (startvolume 10 L), which contained following medium:

TABLE 3
Medium composition seed fermenter

| | | Concentration |
|---|---|---|
| Raw material | | (g/l) |
| Ammonium sulphate | (NH₄)₂SO₄ | 1.0 |
| Potassium dihydrogen phosphate | KH₂PO₄ | 10 |
| Magnesium sulphate | MgSO₄•7H₂O | 5.0 |
| Trace element solution | | 8.0 |
| Vitamin solution | | 8.0 |

The pH was controlled at 5.0 by addition of 28% ammonia. Temperature was controlled at 30° C. pO₂ was controlled at 20% by adjusting the stirrer speed. Glucose concentration was kept limited by controlled feed to the fermenter (exponent of 0.1 was applied).

After 70 hours of fermentation 1.5 L of seed fermenter was transferred to a production fermenter (startvolume 15 L), which contained the following medium:

TABLE 4
Medium composition production fermenter

| Raw material | | Concentration (g/l) |
|---|---|---|
| Ammonium sulphate | (NH₄)₂SO₄ | 2.5 |
| Potassium dihydrogen phosphate | KH₂PO₄ | 3.0 |
| Magnesium sulphate | MgSO₄•7H₂O | 0.5 |
| Trace element solution | | 1 |
| Biotin | | 0.001 |

In the first phase of the fermentation, the pH was controlled at 5.0 by addition of 6 N KOH. After 180 ml 6 N KOH was added to the fermenter, the pH control was released. The pH at end of fermentation was about pH 3. Temperature was controlled at 30° C. Glucose concentration was kept limited by controlled feed to the fermenter (0-24 h: 3.2 g/L/h; >24 h: 2.1 g/L/h).

Three different production fermentations as described above were carried out in the presence of three different gas flows:

During fermentation 1) 0.33 vvm of 100% air was sparged to the fermenter;

During fermentation 2) 0.33 vvm of total gas (50% CO₂, 50% air) was sparged to the fermenter;

During fermentation 3), 0.033 vvm of pure (100%) O₂ was supplied to the headspace.

During all three fermentations, the pO₂ was controlled at 5% by adjusting the stirrer speed.

Results

The succinic acid yield (Yₚₛ) under a 100% O₂ atmosphere was almost twice as high as compared to the yield under 100% air, and similar to the yield under excess CO₂ conditions (Table 5).

TABLE 5
Effect of gas inflow composition on succinic
acid production performance (Yₚₛ),
measured after 42 h of fermentation

| Condition | Yₚₛ (g/g) |
|---|---|
| 0% CO₂, 100% air | 0.24 |
| 50% CO₂, 50% air | 0.41 |
| 100% O₂ | 0.40 |

1. A process for producing a dicarboxylic acid comprising fermenting a microorganism in a suitable fermentation medium wherein a gas flow comprising from 30% to 100% v/v oxygen as measured under atmospheric pressure is added to the fermentation medium and produces dicarboxylic acid.

2. A process according to claim 1, wherein the gas flow comprising from 30% to 100% v/v oxygen is added to said fermentation medium in a continuous way.

3. A process according to claim 2, wherein said process is carried out at a partial pressure of oxygen (O₂) ranging from 0% to 10% v/v.

4. A process according to claim 1, wherein said microorganism is a yeast, optionally a Saccharomyces.

5. A process according to claim 1, wherein said microorganism is a recombinant microorganism.

6. A process according to claim 1, wherein said recombinant microorganism overexpresses a gene encoding an enzyme selected from the group consisting of a phosphoenolpyruvate carboxykinase, malate dehydrogenase, a fumarase, a (NAD(H)-dependent fumarate reductase, a pyruvate carboxylase and a dicarboxylic acid transporter protein.

7. A process according to claim 1, wherein said dicarboxylic acid is malic acid, fumaric acid, succinic acid or adipic acid.

8. A process according to claim 1, wherein said fermenting is carried out at a pH of from 2 to 7.

9. A process according to claim 1, comprising recovering dicarboxylic acid from the fermentation medium.

10. A process according to claim 9, comprising using said dicarboxylic acid in a pharmaceutical, cosmetic, food and/or feed product.

11. A process according to claim 9, wherein said dicarboxylic acid is converted into a polyester polymer.

12. A process according to claim 1, wherein said process is carried out on an industrial scale.

13. A gas flow comprising from 30% to 100% v/v oxygen that has been used for producing a dicarboxylic acid by fermenting a microorganism in a suitable fermentation medium.

14. A method for using a gas flow comprising: generating a 30 to 100% v/v flow of oxygen, using said flow to ferment a microorganism in a suitable fermentation medium to thereby produce a dicarboxylic acid.
